(19) United States
(12) Patent Application Publication
(10) Pub. No.: US 2019/0167706 A1
(43) Pub. Date: Jun. 6, 2019

(54) Solid Pharmaceutical Composition Comprising Amorphous Sofosbuvir

(71) Applicant: Sandoz AG, Basel (CH)

(72) Inventors: Stefano Seminara, Kundl/Tirol (AT); Agnieszka Tajchert, Kundl/Tirol (AT); Franz Schwarz, Kundl/Tirol (AT)

(73) Assignee: Sandoz AG, Basel (CH)

(21) Appl. No.: 16/324,674

(22) PCT Filed: Aug. 9, 2017
(86) PCT No.: PCT/EP2017/070215

§ 371 Date: Feb. 11, 2019

(30) Foreign Application Priority Data
Aug. 12, 2016 (EP) 16184053.3

(51) Int. Cl. A61K 31/7072; A61K 9/14; A61K 9/20

(52) U.S. Cl. CPC A61K 31/7072; A61K 9/145; A61K 9/146; A61K 9/2009; A61K 9/2054; A61K 9/2018; A61K 9/143

(57) ABSTRACT

It is provided a solid pharmaceutical composition comprising sofosbuvir, one or more pharmaceutically acceptable excipients and optionally at least one pharmaceutically acceptable matrix compound, wherein at least 99 weight-% of the sofosbuvir comprised in the solid pharmaceutically composition are present in amorphous form. Sofosbuvir amorphous form is stable in the solid pharmaceutical composition.

The present invention relates to a solid pharmaceutical composition comprising amorphous sofosbuvir and a process for the preparation of the solid pharmaceutical composition. Further, the present invention relates to the use of the solid pharmaceutical composition for the treatment of hepatitis C.

Sofosbuvir according to formula (I)

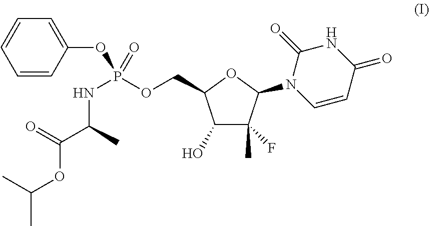 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

with IUPAC name (S)-isopropyl 2-(((S)-(((2R,3R,4R,5R)-5-(2,4-dioxo-3,4-dihydropyrimidin-1(2H)-yl)-4-fluoro-3-hydroxy-4-methyltetrahydrofuran-2-yl)methoxy)(phenoxy)phosphoryl)amino)propanoate is a drug inhibiting the RNA polymerase used by the hepatitis C virus to replicate its RNA.

In WO 2010/135569 A, sofosbuvir is described as a moisture unstable compound. In particular, it was found that under stress conditions at 40° C. and a relative humidity (RH) of 75%, sofosbuvir deliquesces after a few hours. Amorphous sofosbuvir, compared to its crystalline forms, is even less moisture stable and deliquesces at a relative humidity above about 50%. On the other hand, compared to its crystalline forms, amorphous sofosbuvir is believed to show a higher solubility when applied to a patient.

Among many other drugs, WO 2013/101550 A describes sofosbuvir, referred to as PSI-7977. In particular, this document relates to a theoretical assessment tool allegedly useful to rank the intrinsic physical stability of amorphous drug substances. As parameter which indicates the physical stability, the crystallization tendency is mentioned. Without giving any details regarding the specific type of drug, WO 2013/101550 A discloses allegedly stable compositions which may contain from 1 to 50% by weight of the drug wherein, however, the drug content is preferably in the range of from 5 to 15% by weight. Not one single actual example directed to a concrete composition which would have been subjected to a respective stability test is disclosed in WO 2013/101550 A. Still further, theoretical examples according to WO 2013/101550 A directed to HCV inhibitors in general teach a very low drug content of only 10% by weight.

Therefore, the problem underlying the present invention is the provision of a stable pharmaceutical composition comprising amorphous sofosbuvir wherein the amorphous sofosbuvir is stabilized.

It has now been surprisingly found that amorphous sofosbuvir formulated in a solid dosage form without the need of preparing a solid dispersion comprising sofosbuvir and a matrix compound is stable. It has been found that amorphous sofosbuvir is stable in a solid pharmaceutical composition wherein the solid pharmaceutical composition is prepared from a dry mixture comprising amorphous sofosbuvir, at least one pharmaceutically acceptable matrix compound and at least one pharmaceutically acceptable excipient. The solid pharmaceutical composition is preferably prepared by melt extrusion, more preferably by hot melt extrusion of said mixture or by directed spray drying of sofosbuvir with a matrix compound or alone, preferably sofosbuvir alone to amorphous sofosbuvir and then admixing it at least one pharmaceutically acceptable excipient.

It has been further found that amorphous sofosbuvir is stable in a solid pharmaceutical composition wherein the solid pharmaceutical composition is prepared from a mixture comprising amorphous sofosbuvir and at least one pharmaceutically acceptable excipient. The stability of amorphous sofosbuvir is further improved when the amorphous sofosbuvir is prepared by spray drying.

Hence advantageously, it is not necessary to take into account the stability of amorphous sofosbuvir in the solid pharmaceutical composition of the invention since it was found that in the solid pharmaceutical composition of the invention, sofosbuvir is stable in its amorphous form.

Advantageously, the process for preparing the solid pharmaceutical is quite simplified since sofosbuvir, the at least one pharmaceutically acceptable matrix compound, when present, and the one or more pharmaceutically acceptable excipient(s) are mixed together in one pot.

Advantageously, the particle size and the particle distribution of amorphous sofosbuvir—when spray dried with or without the matrix compound-can be controlled. Advantageously, the solvent is completely removed. The process allows standardizing the particle distribution and allows complying with the requirement of the US pharmacopeia concerning the residual solvent. Further, the solid pharmaceutical composition of the invention show good pharmacokinetic properties and bioavailability.

The present invention relates to a solid pharmaceutical composition comprising sofosbuvir according to formula (I)

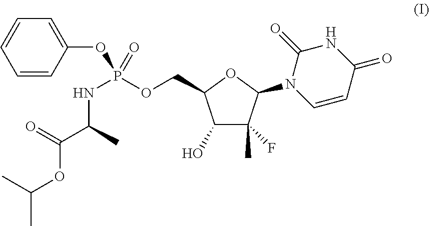 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s).

According to the invention, it is preferred that the at least one pharmaceutically acceptable matrix compound comprises, preferably consists of, at least one vinyl pyrrolidone-vinyl acetate copolymer wherein the at least one vinyl pyrrolidone-vinyl acetate copolymer is copovidone.

Further, the present invention relates to a process for preparing a solid pharmaceutical composition of the invention, the process comprising
(i) preparing a mixture comprising amorphous sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
(ii) processing the mixture obtained from (i) to the solid pharmaceutical composition.

Further, the present invention relates to a process for preparing a solid pharmaceutical composition of the invention, the process comprising
(i′) preparing a mixture of sofosbuvir and the at least one pharmaceutically acceptable matrix compound and at least one solvent;
(ii′) subjecting the mixture of (i′) to drying, preferably by lyophilizing the mixture of (i′) or spray-drying the mixture of (i′), obtaining a dried, preferably lyophilize or spray-dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition.

Further, the present invention relates to a solid pharmaceutical composition wherein the solid pharmaceutical composition comprises sofosbuvir according to formula (I)

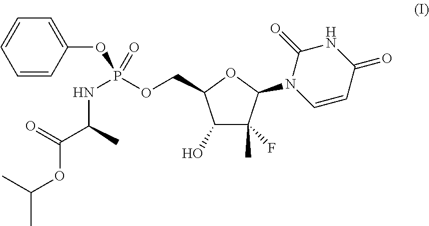 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir and the one or more pharmaceutically acceptable excipient(s).

Amorphous sofosbuvir is preferably formed in step (ii′)

Further, the present invention relates a process for preparing said solid pharmaceutical composition, the process comprising:
(i) preparing amorphous sofosbuvir, wherein the preparing is preferably carried out by rapid drying, more preferably by spray drying a solution comprising sofosbuvir and one or more solvents;
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

Further, the present invention relates to a solid pharmaceutical composition obtained or obtainable according to any one of the processes according to the invention.

Further, the present invention relates to a tablet obtained or obtainable according to any one of the processes according to the invention.

Further, the present invention relates to a solid pharmaceutical composition according the invention for the preparation of a medicament for treating hepatitis C in a human.

Further, the present invention relates to a method for treating hepatitis C comprising administering a solid pharmaceutical composition according to the invention to a human in need thereof.

Solid Pharmaceutical Composition

Therefore the present invention is directed to a solid pharmaceutical composition wherein the solid pharmaceutical composition comprises sofosbuvir according to formula (I)

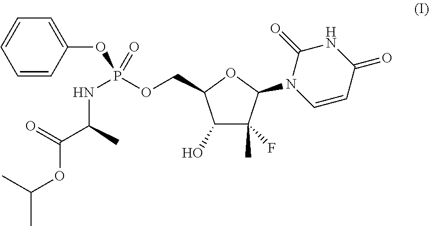 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s).

Preferably the solid pharmaceutical composition according to the invention comprises sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.

Preferably from 20 to 45 weight-% or from 20 to 45 weight-% or from 30 to 40 weight-% of the solid pharmaceutical composition of the invention consists of sofosbuvir based on the total weight of the solid pharmaceutical composition. More preferably 30 weight-% of the solid pharmaceutical composition consists of sofosbuvir based on the total weight of the solid pharmaceutical composition.

Preferably, according to the present invention, at least 99 weight-% of the sofosbuvir comprised in the solid pharmaceutical composition are present in amorphous form. Preferably, at least 99.5 weight-%, more preferably at least 99.6 weight-%, more preferably at least 99.7 weight-%, more preferably at least 99.8 weight-%, more preferably at least 99.9 weight-% of the sofosbuvir comprised in the solid composition are present in amorphous form. More preferably, at least 99.95 weight-%, more preferably at least 99.99 weight-% of the sofosbuvir comprised in the solid composition are present in amorphous form. The term “amorphous form” as used in this context of the present invention relates to sofosbuvir which, subjected to X-ray powder diffraction spectroscopy, does not contain any peaks related to crystalline form of sofosbuvir.

Therefore the present invention is preferably directed to a solid pharmaceutical composition wherein the solid pharmaceutical composition comprises sofosbuvir according to formula (I)

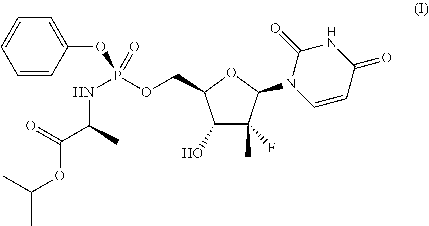 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99.5 weight-%, preferably 99.9 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s) and

Matrix Compound

According to the invention, the at least one pharmaceutically acceptable matrix compound is preferably selected from the group consisting of hydrophilic polymers, silicon-based inorganic adsorbents and a combination of two or more thereof. The at least one pharmaceutically acceptable matrix compound is optionally comprised in the solid pharmaceutical composition of the invention.

Regarding the at least one pharmaceutically acceptable matrix compound, it was found that in particular hydrophilic polymers, preferably hydrophilic water-soluble polymers, and silicon-based inorganic adsorbents are suitable matrix compounds. Preferably, the at least one matrix compound is selected from the group consisting of hydrophilic water-soluble polymers, silicon-based inorganic adsorbents and a combination of two or more thereof. For example, the at least one matrix compound is selected from the group consisting of hydrophilic polymers, preferably hydrophilic water-soluble polymers, and combinations of two or more thereof; or from the group consisting of silicon-based inorganic adsorbents and combinations of two or more thereof; or from the group consisting of combinations of at least one hydrophilic polymer, preferably hydrophilic water-soluble polymer, and at least one silicon-based inorganic adsorbent.

a) Silicon-Based Inorganic Adsorbents

Regarding the silicon-based inorganic adsorbents they include, preferably are, one or more of silica and silicates. Preferably the at least one silicon-based inorganic adsorbent has an oil absorbance in the range of from 1.0 to 5.0 ml/g, preferably in the range of from 1.5 to 4.0 ml/g. the at least one silicon-based inorganic adsorbent has a bulk density in the range of from 10 to 600 g/ml, preferably in the range of from 30 to 500 g/ml, more preferably in the range of from 50 to 300 g/ml, more preferably in the range of from 50 to 200 g/ml. Preferably the at least one silicon-based inorganic adsorbent is selected from the group consisting of silica, silicates, and a combination of two or more thereof, wherein the silica is preferably selected from the group consisting of fumed silica, precipitated silica, gel silica, colloidal silica, and a combination of two or more thereof, and wherein the silicates are preferably aluminosilicates preferably comprising at least one alkali metal element and/or at least one alkaline earth metal element, more preferably at least one alkaline earth metal element, more preferably magnesium, wherein more preferably, at least 90 weight-%, more preferably at least 95 weight-%, more preferably at least 99 weight-% of the at least one silicon-based inorganic adsorbent are present in amorphous form.

Examples of silicon-based inorganic adsorbents include, but are not restricted to, silica, silicates, and a combination of two or more thereof. For example, the silicon-based inorganic adsorbent is selected from the group consisting of silicas and combinations of two or more thereof; or from the group consisting of silicates and combinations of two or more thereof; or from the group consisting of at least one silica and at least one silicate. The term “silicate” as used in this context of the present invention refers to naturally occurring or synthesized compounds containing an anionic silicon compound, preferably an oxide. Examples of such silicates include, but are not restricted to, nesosilicates comprising the structure unit [SiO₄]⁴ sorosilicates comprising the structure unit [Si₂O₇]⁶⁻, cyclosilicates comprising the structure unit [SiₙO₃ₙ]²ⁿ⁻, single chain inosilicates comprising the structure unit [SiₙO₃ₙ]²ⁿ⁻, double chain inosilicates comprising the structure unit [Si₄ₙO₁₁ₙ]⁶ⁿ⁻, phyllosilicates comprising the structure unit [SiₙO₅ₙ]²ⁿ⁻, or tectosilicates with a 3D framework comprising the structure unit [AlₓSi_{y}O_{2(x+y)}]^{x-}. The term “silica” as used in this context of the present invention refers to naturally occurring or synthesized silica. Examples of such silica include, but are not restricted to fumed silica, precipitated silica, gel silica, colloidal silica, such as Syloid® AL-1 FP and Syloid 72FP silica, Syloid 244 FP silica, Syloid 74FP silica, Syloid 63FP silica or Aerosil.

b) Hydrophilic Polymers

Regarding the hydrophilic polymers, they include, preferably are, one or more of polysaccharides, preferably cellulose derivatives such as hydroxyalkylalkylcelullose, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.

Regarding the hydrophilic polymers, they are preferably selected from the group consisting of hydrophilic water-soluble polymers and a combination of two or more thereof. Preferably the hydrophilic water-soluble polymer has a solubility in water of at least 10 g/l, preferably of at least 20 g/l, more preferably of at least 30 g/l, in each case at 23° C. at atmospheric pressure. It is preferred that the weight average molecular weight (M_{w}) of the at least one hydrophilic water-soluble polymer is in the range of from 20 to 100 kDa, preferably in the range of from 30 to 85 kDa, more preferably in the range of from 40 to 80 kDa.

Preferably the hydrophilic water-soluble polymer are selected from the group consisting of polyvinylpyrrolidones (PVP, polyvidone, povidone) such as PVP 40, vinyl pyrrolidone-based copolymers such as vinyl pyrrolidone-vinyl acetate copolymer like copovidone, and other polymers such as polyethylene glycol, polyvinyl acetate and polyvinylcaprolactame-based graft copolymer like Soluplus®.

More preferably the hydrophilic water-soluble polymer comprises, preferably consists of, a vinyl pyrrolidone-vinyl acetate copolymer, wherein preferably the vinyl pyrrolidone-vinyl acetate copolymer comprises, preferably consists of, copovidone.

Regarding the hydrophilic polymers, the at least one hydrophilic water-soluble polymer may comprises, preferably consists of a cellulose derivative selected from the group consisting of hydroxyalkylalkylcelluloses and a mixture of two or more thereof, the at least one hydrophilic water-soluble polymer preferably comprising, more preferably consisting of, hydroxypropylmethylcellulose (HPMC). Preferably, the cellulose derivative has a degree of substitution (DS) in the range of from 0.3 to 2.8, more preferably in the range of from 0.6 to 2.5, more preferably in the range of from 1.0 to 2.3, more preferably in the range of from 1.3 to 2.0. Preferably, the weight average molecular weight (M_{w}) of the cellulose derivative is in the range of from 7 to 225 kDa, more preferably in the range of from 7 to 100 kDa, more preferably in the range of from 7 to 30 kDa.

More specifically cellulose derivatives are preferably selected from the group consisting of alkylcellulose, preferably methylcellulose, ethylcellulose, or propylcellulose; hydroxalkylcellulose, preferably hydroxymethylcellulose, hydroxyethylcellulose, or hydroxypropylcellulose (HPC) such as Klucel® LF; hydroxyalkylalkylcellulose, preferably hydroxyethylmethylcellulose (HEMC), or hydroxypropylmethylcellulose (HPMC); carboxyalkylcellulose, preferably carboxymethylcellulose (CMC), carboxymethylhydroxyethylcellulose (CMHEC), hydroxyethylcarboxymethylcellulose (HECMC); sodium carboxymethylcellulose, cellulose acetate phthalate (CAP), hydroxypropylmethylcellulose acetate (HPMCA), hydroxypropylmethylcellulose phthalate (HPMCP), hydroxypropylmethylcellulose acetate succinate (HPMCAS), and a mixture of two or more thereof.

Therefore the present invention is preferably directed to a solid pharmaceutical composition wherein the solid pharmaceutical composition comprises sofosbuvir according to formula (I)

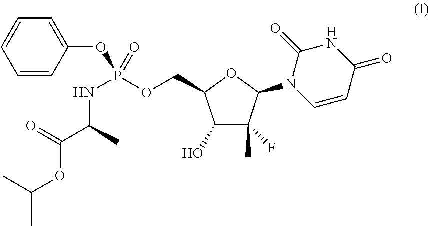 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s), wherein 20 to 45 weight-%, more preferably from 20 to 40 weight-%, more preferably from 30 to 40 weight-%, more preferably 30 weight-% of the solid pharmaceutical composition consists of sofosbuvir based on the total weight of the solid pharmaceutical composition and wherein at least one pharmaceutically acceptable matrix compound comprises, preferably consists of a vinyl pyrrolidonevinyl acetate copolymer, wherein preferably the vinyl pyrrolidone-vinyl acetate copolymer comprises, preferably consists of, copovidone.

According to the present invention, at least 99 weight-% of the solid pharmaceutical composition consists of the sofosbuvir, the at least one matrix compound and the one or more pharmaceutically acceptable excipient(s). Preferably, at least 99.5 weight-%, more preferably at least 99.6 weight-%, more preferably at least 99.7 weight-%, more preferably at least 99.8 weight-%, more preferably at least 99.9 weight-% of the solid composition consists of the sofosbuvir, the at least one matrix compound and the one or more pharmaceutically acceptable excipient(s). More preferably, at least 99.95 weight-%, more preferably at least 99.99 weight-% of the solid composition consists of the sofosbuvir, the at least one matrix compound and the one or more pharmaceutically acceptable excipient(s).

According to the invention, preferably from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.

It is contemplated that the solid pharmaceutical composition of the invention can be a solid dispersion. The term “solid dispersion” as used in this context of the present invention relates to a composition in a solid state, i.e. a state which is neither liquid nor gaseous, wherein the amorphous sofosbuvir is dispersed in at least one of the at least one pharmaceutically acceptable matrix compounds and the one or more pharmaceutically acceptable excipient(s) comprised in the solid dispersion, preferably in all of the at least pharmaceutically acceptable one matrix compounds and the one or more pharmaceutically acceptable comprised in the solid dispersion.

Preferably, the solid pharmaceutical composition of the present invention is an oral dosage form, including, but not restricted to, a granule, a capsule, for example a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet or an extrudate. More preferably, the oral dosage form of the present invention is a tablet.

It is contemplated that the solid pharmaceutical composition of the invention comprises, in addition to the sofosbuvir, one or more further HCV agents. The one or more further HCV agents can include one or more of ledipasvir according to formula (II)

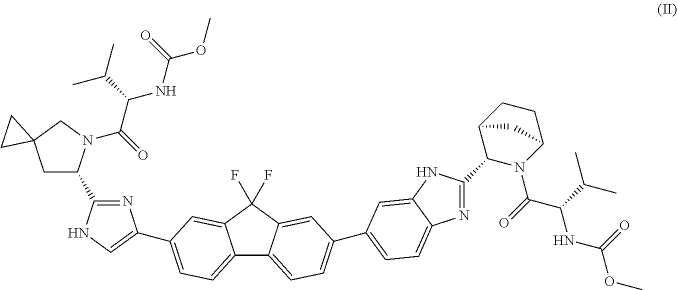 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

and daclatasvir of formula (III)

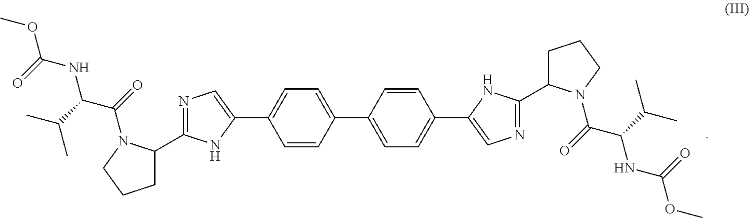

Preferably, the solid pharmaceutical composition of the invention consists of sofosbuvir, the at least one pharmaceutically acceptable matrix compound, the one or more pharmaceutically acceptable excipient(s) and optionally the one or more further HCV agents.

One or More Pharmaceutically Acceptable Excipient(s)

The terms “pharmaceutically acceptable excipient” and “pharmaceutical excipient” as used in this context of the present invention refer to a compound that is used to prepare a pharmaceutical composition, and is generally safe, non-toxic and neither biologically nor otherwise undesirable, and includes excipients that are acceptable for veterinary use as well as human pharmaceutical use.

According to the present invention, it is preferred that at least one of the one or more pharmaceutically acceptable excipient(s) is different from the at least one pharmaceutically acceptable matrix compound comprised in the solid pharmaceutical composition. Hence it is preferred that the at least one of the one or more pharmaceutically acceptable excipient(s) is not a pharmaceutically acceptable matrix compound as defined above. Hence for example, the excipient is not povidone. For example the excipient is not PLASDONE™ S-630 or polyvinylpyrrolidine K-30. Hence, for example if the solid pharmaceutical composition of the invention comprises povidone such as PLASDONE™ S-630 or polyvinylpyrrolidine K-30 as pharmaceutically acceptable matrix compound, the composition of the invention further comprises one or more excipients that are not povidone such as PLASDONE™ S-630 or polyvinylpyrrolidine K-30.

Generally there is no particular restriction as to the amount of the one or more pharmaceutically acceptable excipient(s) comprised in the solid pharmaceutical composition of the invention. Preferably at least 1.5 weight-% more preferably from 10 to 85 weight-%, more preferably from to 30 to 80 weight-%, more preferably from 40 to 70 weight-, more preferably from 50 to 60 of the solid pharmaceutical composition consists of the one or more pharmaceutically acceptable excipients based on the total weight of the of the solid pharmaceutical composition.

According to the present invention, it is contemplated that the one or more pharmaceutically acceptable excipient(s) comprise one or more of at least one of a diluent, at least one disintegrant, at least one glidant, optionally at least one lubricant, and combinations of two or more thereof.

a) Diluent

Regarding the at least one diluent, it is preferably selected from the group consisting of one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate. More preferably, the diluent is mannitol

It is preferred that the solid pharmaceutical composition of the invention comprises from 20 to 30 weight-%, more preferably from 22 to 26 weight-% diluent, based on the total weight of the solid pharmaceutical composition. It is preferred that the solid pharmaceutical composition of the invention comprises from 20 to 30 weight-%, more preferably from 22 to 26 weight-% of mannitol, based on the total weight of the solid pharmaceutical composition.

It is preferred that at least 15 weight-%, more preferably from 20 to 30 weight-%, more preferably from 22 to 26 weight-% of the solid pharmaceutical composition of the invention consists of the diluent, based on the total weight of the solid pharmaceutical composition. It is preferred that at least 15 weight-%, more preferably from 20 to 30 weight-%, more preferably from 22 to 26 weight-% of the solid pharmaceutical composition of the invention consists of the diluent wherein the diluent comprises, preferably is mannitol, based on the total weight of the solid pharmaceutical composition.

b) Disintegrant

Regarding the at least one disintegrant, it is preferably selected from the group consisting of one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates. Silicon dioxide and silica are used interchangeably in the context of the invention.

More preferably, the disintegrant is selected from croscarmellose sodium, such as Ac-Di-Sol® croscarmellose sodium.

It is preferred that at least 16.5 weight-%, more preferably from 22 to 36 weight-%, more preferably from 27.5 to 32.5 weight-% of the solid pharmaceutical composition of the invention consists of the disintegrant, based on the total weight of the solid pharmaceutical composition. It is preferred at least 16.5 weight-%, more preferably from 22 to 36 weight-%, more preferably from 27.5 to 32.5 weight-% of that the solid pharmaceutical composition of the invention consists a disintegrant wherein the disintegrant comprises, preferably is croscarmellose sodium or microcrystalline cellulose or mixture thereof, based on the total weight of the solid pharmaceutical composition.

It is preferred that the disintegrant comprises a first and a second disintegrant, more preferably consists of a first and a second disintegrant.

It is preferred that the solid pharmaceutical composition of the invention comprises from 25 to 40 weight-%, preferably from 25 to 30 weight-% disintegrant, based on the total weight of the solid pharmaceutical composition. It is preferred that the solid pharmaceutical composition of the invention comprises from 25 to 40 weight-%, preferably from 25 to 30 weight-% disintegrant of croscarmellose sodium based on the total weight of the solid pharmaceutical composition.

It is preferred that at least 15 weight-%, more preferably from 25 to 40 weight-%, more preferably from 25 to 30 weight-% of the solid pharmaceutical composition of the invention consists of the first disintegrant wherein the first disintegrant is preferably microcrystalline cellulose or croscarmellose sodium based on the total weight of the solid pharmaceutical composition.

The solid pharmaceutical composition of the invention can comprise a first disintegrant and a second disintegrant that can be the same disintegrant or different disintegrant It is preferred that at least 1.5 weight-% more preferably from 2 to 6 weight-%, more preferably from 2.5 to 5.5 weight-% of the solid pharmaceutical composition of the invention consists of a the second disintegrant, based on the total weight of the solid pharmaceutical composition. It is preferred that the second disintegrant comprises, more preferably is croscarmellose sodium.

c) Glidant

Regarding the at least one glidant it is preferably selected from the group consisting of one or more of colloidal silicon dioxide, talc, starch, starch derivatives. More preferably, the glidant is colloidal silicon dioxide.

It is preferred that the solid pharmaceutical composition of the invention from 0.5 to 7 weight-%, preferably from 0.8 to 5 weight-% glidant, based on the total weight of the solid pharmaceutical composition. It is preferred that the solid pharmaceutical composition of the invention comprises from 0.5 to 7 weight-%, preferably from 0.8 to 5 weight-% of colloidal silicon dioxide, based on the total weight of the solid pharmaceutical composition.

It is preferred that at least 0.2 weight-%, more preferably from 0.5 to 7 weight-%, more preferably from 0.8 to 5 weight-% of the solid pharmaceutical composition consists of the glidant, based on the total weight of the solid pharmaceutical composition. It is preferred that at least 0.2 weight-%, more preferably from 0.5 to 7 weight-%, more preferably from 0.8 to 5 weight-% of the solid pharmaceutical composition consists of the glidant wherein the glidant comprises, preferably is colloidal silicon dioxide, based on the total weight of the solid pharmaceutical composition.

d) Lubricant

Regarding at least one lubricant, it is preferably selected from the group consisting of one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.

More preferably, the lubricant is selected from the group consisting of magnesium stearate.

It is preferred that the solid pharmaceutical composition of the invention comprises from 1 to 6 weight-%, preferably from 1.4 to 2 weight-% lubricant based on the total weight of the solid pharmaceutical composition. It is preferred that the solid pharmaceutical composition of the invention comprises from 1 to 6 weight-%, preferably from 1.4 to 2 weight-% magnesium stearate, based on the total weight of the solid pharmaceutical composition.

It is preferred that at least 0.5 weight-%, more preferably from 1 to 6 weight-%, more preferably from 1.4 to 2 weight-% of the solid pharmaceutical composition of the invention consists of the lubricant based on the total weight of the solid pharmaceutical composition. It is preferred that at least 0.5 weight-%, more preferably from 1 to 6 weight-%, more preferably from 1.4 to 2 weight-% of the solid pharmaceutical composition of the invention consists of the lubricant, wherein the lubricant comprises, preferably is magnesium stearate, based on the total weight of the solid pharmaceutical composition.

Additional Excipients

It is contemplated that the composition of the invention may comprise any other suitable additional excipients known to the skilled person, if the addition of said additional excipients is required. Additional excipients are for example buffering agents, emulsifiers, wetting agents, suspending agents, sweetening agents, colorants, flavors, coating agents, preservatives, antioxidants, processing agents, drug delivery modifiers, additives to make solutions isotonic, antifoaming agents, encapsulating material, surfactants, opacifing agents, enhancers, waxes, cap anti-locking agents (e.g. glycerol) and ion exchange resins. Other conceivable pharmaceutically acceptable additives are described in Remington's Pharmaceutical Sciences, 15^{th} edition, Mack Publishing Co., New Jersey (1991).

Conceivably, the pharmaceutical composition of the present invention, in particular in form of a tablet, may further comprise a coating agent which may further comprise a taste-masking agent. The coating agent may be formed from an aqueous film coat composition, wherein the aqueous film coat composition may comprise a film-forming polymer, water and/or an alcohol as a vehicle, and optionally one or more adjuvants such as are known in the film-coating art. The coating agent may be selected from among hydroxypropylmethylcellulose, hydroxypropylcellulose, methylcellulose, ethylcellulose, hydroxyethylcellulose, cellulose acetate phthalate, sodium ethyl cellulose sulfate, carboxymethyl cellulose, polyvinylpyrrolidone, zein, and an acrylic polymer such as methacrylic acid/methacrylic acid ester copolymers such as methacrylic acid/methylmethacrylate copolymers, etc., and a polyvinyl alcohol. With respect to the coating agent, film-forming polymers are typically provided in either aqueous or organic solvent-based solutions or aqueous dispersions. The polymers may be also provided in dry form, alone or in a powdery mixture with other components such as a plasticizer and/or a colorant, which may be made into a solution or dispersion. The aqueous film coat composition may further comprise water as a vehicle for the other components. The vehicle may optionally further comprise one or more water soluble solvents, such as an alcohol and/or a ketone. Conceivable examples of an alcohol include but are not limited to methanol, isopropanol, propanol, etc. A non-limiting example for the ketone may be acetone. Suitable aqueous film coating compositions may include those commercially available from Colorcon, Inc. of West Point, Pa., under the trade name OPADRY and OPADRY II.

It is contemplated that the one or more pharmaceutically acceptable excipient(s) comprise, preferably are, a combination of at least one a diluent, at least one disintegrant, at least one glidant, and optionally at least one lubricant.

Therefore, the solid pharmaceutical composition of the invention preferably comprises
from 20 to 30 weight-%, preferably from 22 to 26 weight-% diluent,
from 20 to 30 weight-%, preferably from 24 to 28 weight-% first disintegrant,
from 2 to 6 weight-%, preferably from 2.5 to 5.5 weight-% second disintegrant,
from 0.5 to 7 weight-%, preferably from 0.8 to 5 weight-% glidant,
optionally from 1 to 6 weight-%, preferably from 1.4 to 2 weight-% lubricant,
   in each based on the total weight of the solid pharmaceutical composition.

Therefore, the solid pharmaceutical composition of the invention preferably comprises
from 30 to 45 weight-%, preferably from 30 to 40 weight-% sofosbuvir,
from 3 to 15 weight-%, preferably from 3 to 13 weight-% of the pharmaceutically acceptable matrix compound,
from 20 to 30 weight-%, preferably from 22 to 26 weight-% diluent,
from 20 to 30 weight-%, preferably from 24 to 28 weight-% first disintegrant,
from 2 to 6 weight-%, preferably from 2.5 to 5.5 weight-% second disintegrant,
from 0.5 to 7 weight-%, preferably from 0.8 to 5 weight-% glidant,
optionally from 1 to 6 weight-%, preferably from 1.4 to 2 weight-% lubricant,
   in each based on the total weight of the solid pharmaceutical composition.

It is further preferred that the solid pharmaceutical composition wherein the one or more pharmaceutically acceptable excipient(s) comprise, preferably are, a combination of mannitol, microcrystalline cellulose, croscarmellose sodium, silica dioxide and optionally magnesium stearate.

Therefore, the solid pharmaceutical composition of the invention preferably comprises
from 20 to 30 weight-%, preferably from 22 to 26 weight-% mannitol,
from 20 to 30 weight-%, preferably from 24 to 28 weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
optionally from 1 to 6 weight-%, preferably from 1.4 to 2 weight-% magnesium stearate,
   in each based on the total weight of the solid pharmaceutical composition.

The present invention is further preferably directed to a solid pharmaceutical composition comprising amorphous sofosbuvir, the at least one pharmaceutically acceptable matrix compound, wherein the least one pharmaceutically acceptable matrix compound preferably is copovidone, and the one or more pharmaceutically acceptable excipient(s), wherein the one or more pharmaceutically acceptable excipient(s) comprise, preferably are, a combination of mannitol, microcrystalline cellulose, croscarmellose sodium, silicon dioxide, and optionally magnesium stearate.

Therefore, the solid pharmaceutical composition of the invention preferably comprises
from 30 to 45 weight-%, preferably from 30 to 40 weight-% amorphous sofosbuvir,
from 3 to 15 weight-%, preferably from 3 to 13 weight-% more preferably from 3 to 5 weight-% copovidone,
from 20 to 30 weight-%, preferably from 22 to 26 weight-% mannitol,
from 20 to 30 weight-%, preferably from 24 to 28 weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
optionally from 1 to 6 weight-%, preferably from 1.4 to 2 weight-% magnesium stearate,
   in each based on the total weight of the solid pharmaceutical composition.

Therefore, the solid pharmaceutical composition of the invention more preferably comprises
from 30 to 45 weight-% amorphous sofosbuvir,
from 3 to 5 weight-% copovidone,
from 22 to 26 weight-% mannitol,
from 24 to 28 weight-% microcrystalline cellulose,
from 2.5 to 5.5 weight-% croscarmellose sodium,
from 0.8 to 5 weight-% colloidal silica,
optionally from 1.4 to 2 weight-% magnesium stearate,
   in each based on the total weight of the solid pharmaceutical composition, wherein the individual contents add up to 100%.

It is further contemplated that solid pharmaceutical composition as disclosed above is a solid oral dosage form, preferably a tablet.

Therefore, the present invention is directed to a tablet preferably comprising
from 20 to 30 weight-%, preferably from 22 to 26 weight-% mannitol,
from 20 to 30 weight-%, preferably from 24 to 28 weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
optionally from 1 to 6 weight-%, preferably from 1.4 to 2 weight-% magnesium stearate,
   in each based on the total weight of the solid pharmaceutical composition.

Therefore, the present invention is directed to a tablet preferably comprising
from 30 to 45 weight-%, preferably from 30 to 40 weight-% amorphous sofosbuvir,
from 5 to 15 weight-%, preferably from 7 to 13 weight-%, more preferably from 3 to 5 weight-% copovidone,
from 20 to 30 weight-%, preferably from 22 to 26 weight-% mannitol,
from 20 to 30 weight-%, preferably from 24 to 28 weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
optionally from 1 to 6 weight-%, preferably from 1.4 to 2 weight-% magnesium stearate,
   in each based on the total weight of the solid pharmaceutical composition.

Therefore, the present invention is directed to a tablet more preferably comprising
from 30 to 40 weight-% amorphous sofosbuvir,
from 3 to 5 weight-% copovidone,
from 22 to 26 weight-% mannitol,
from 24 to 28 weight-% microcrystalline cellulose,
from 2.5 to 5.5 weight-% croscarmellose sodium,
from 0.8 to 5 weight-% colloidal silica,
optionally from 1.4 to 2 weight-% magnesium stearate,
   in each based on the total weight of the solid pharmaceutical composition, wherein the individual contents add up to 100%.

The tablet according to the invention can be a coated tablet.

It is further contemplated that the solid pharmaceutical composition of the invention, wherein the solid pharmaceutical composition of the invention is preferably a tablet has a weight in the range of from 900 mg to 2300 mg, preferably 1000 mg to 2000 mg.

Certain compositions disclosed in WO 2013/101550 A describe compositions comprising 10% by weight of a drug different from sofosbuvir contain a surfactant, namely vitamin E TPGS, sorbitan monolaurate, propylene glycol monocarpylate, or a combination of vitamin E TGPS and lauryl glycol FCC. These surfactants are disclosed to be present in the compositions in very significant amounts of 7 weight-%, based on the total weight of the compositions. Thus, it appears that WO 2013/101550 A, in its most concrete embodiments, teaches the mandatory use of surfactants in significant amounts if a physically stable composition is to be provided. Surprisingly, for the solid pharmaceutical compositions of the present invention comprising at least 30 weight-% of amorphous sofosbuvir, it was found that no such surfactant is necessary to ultimately provide a physically stable solid pharmaceutical composition. Therefore, the present invention also relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of vitamin E TPGS (D-alpha-tocopheryl polyethylene glycol 1000 succinate), or of sorbitan monolaurate, or of a combination of vitamin E TGPS and lauryl glycol FCC. Preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of polysorbate 20, or of polysorbate 40, or of polysorbate 60, or of polysorbate 80, or of Cremophor RH 40, or of Cremophor EL, or of Gelucire 44/14, or of Gelucire 50/13, or of vitamin E TPGS, or of propylene glycol laurate, or of sodium lauryl sulfate, or of sorbitan monolaurate, or of a combination or a mixture of two or more thereof. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of polyoxyethylene castor oil derivatives, e.g. polyoxyethyleneglycerol triricinoleate or polyoxyl 35 castor oil (Cremophor EL; BASF Corp.) or polyoxyethyleneglycerol oxystearate such as polyethylenglycol 40 hydrogenated castor oil (Cremophor RH 40, also known as polyoxyl 40 hydrogenated castor oil or macrogolglycerol hydroxystearate) or polyethylenglycol 60 hydrogenated castor oil (Cremophor RH 60); or a mono fatty acid ester of polyoxyethylene sorbitan, such as a mono fatty acid ester of polyoxyethylene (20) sorbitan, e.g. polyoxyethylene (20) sorbitan monooleate (Tween 80), polyoxyethylene (20) sorbitan monostearate (Tween 60), polyoxyethylene (20) sorbitan monopalmitate (Tween 40), or polyoxyethylene (20) sorbitan monolaurate (Tween 20), or polyoxyethylene alkyl ethers, e.g. polyoxyethylene (3) lauryl ether, polyoxyethylene (5) cetyl ether, polyoxyethylene (2) stearyl ether, polyoxyethylene (5) stearyl ether; or polyoxyethylene alkylaryl ethers, e.g. polyoxyethylene (2) nonylphenyl ether, polyoxyethylene (3) nonylphenyl ether, polyoxyethylene (4) nonylphenyl ether, polyoxyethylene (3) octylphenyl ether; or polyethylene glycol fatty acid esters, e.g. PEG-200 monolaurate, PEG-200 dilaurate, PEG-300 dilaurate, PEG-400 dilaurate, PEG-300 distearate, PEG-300 dioleate; alkylene glycol fatty acid mono esters, e.g. propylene glycol monolaurate (lauroglycol, such as lauroglycol FCC); or sucrose fatty acid esters, e.g. sucrose monostearate, sucrose distearate, sucrose monolaurate, sucrose dilaurate; or sorbitan fatty acid mono esters such as sorbitan mono laurate (Span 20), sorbitan monooleate, sorbitan monopalmitate (Span 40), or sorbitan stearate; or D-alpha-tocopheryl polyethylene glycol 1000 succinate; or a combination or mixture thereof; or block copolymers of ethylene oxide and propylene oxide, also known as polyoxyethylene polyoxypropylene block copolymers or polyoxyethylene polypropyleneglycol, such as Poloxamer 124, Poloxamer 188, Poloxamer 237, Poloxamer 388, or Poloxamer 407, or a combination of two or more thereof. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable surfactant having an HLB value of from 2-20. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable non-ionic surfactant. More preferably, the present invention relates to the above-described solid pharmaceutical composition, wherein the solid composition comprised in the solid pharmaceutical composition comprises less than 0.1 weight-%, preferably less than 0.01 weight-%, more preferably less than 0.001 weight-%, more preferably less than 0.0001 weight-%, more preferably in the range of from 0 to 0.00001 weight-% of a pharmaceutically acceptable surfactant. In each case, the weight-% values are based on the total weight of the solid composition.

It is further contemplated that the solid pharmaceutical composition of the invention as disclosed above is obtainable or obtained by a process comprising
(i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
(ii) processing the mixture obtained from (i) to the solid pharmaceutical composition, wherein preferably the solid pharmaceutical composition is a tablet.

The at least one matrix compound and the one or more pharmaceutically acceptable excipient(s) are as disclosed above. It is preferred that step (ii) is carried out directly. Hence it is contemplated a process wherein
(ii) directly processing the mixture obtained from (i) to the pharmaceutical composition.

It is further preferred that after (i) and before (ii), the mixture obtained from (i) is not subjected to any modification. It is further preferred that the mixture obtained from (i) is processed to the pharmaceutical composition according to (ii) at most 168 h, preferably at most 72 h, more preferably at most 24 h after having been obtained from (i), wherein during this period of time, the mixture is preferably not subjected to stress conditions of 30° C. and a relative humidity of 75%, more preferably stored under ambient conditions.

According to the present invention, the term “ambient conditions” refers to a temperature of 25° C. and an atmospheric pressure of 100 kPa.

It is further preferred that the processing of (ii) comprise, preferably is one or more of wet granulation, dry granulation, compression, melting extrusion of the mixture of (i). More preferably (ii) comprises, preferably consists in melting extruding the mixture of (i), more preferably hot melting extruding the mixture of (i). Preferably, the melt extrusion is carried out at a temperature of at least 100° C., preferably at least 150° C.

Therefore the present invention is directed to a solid pharmaceutical composition obtainable or obtained by a process comprising
(i) preparing a mixture comprising sofosbuvir, at least one pharmaceutically acceptable matrix compound and one or more pharmaceutically acceptable excipient(s), wherein at least 99 weight-% preferably at least 99.9 weight-% of the mixture consists of the sofosbuvir, the at least one matrix compound and the one or more pharmaceutically acceptable excipient(s);
(ii) processing the mixture obtained from (i) to the solid pharmaceutical composition, wherein the processing comprises
   (ii-1) melt extruding preferably hot melting extruding the mixture obtained from (i) and obtaining an extrudate
   (ii-2) working the extrudate of (ii-1) and obtaining to the solid pharmaceutical composition.

Therefore the present invention is directed to a solid pharmaceutical composition obtainable or obtained by a process comprising
(i) preparing a mixture comprising sofosbuvir, a vinyl pyrrolidone-vinyl acetate copolymer, wherein the vinyl pyrrolidone-vinyl acetate copolymer is preferably is copovidone and one or more pharmaceutically acceptable excipient(s), wherein at least 99 weight-%, preferably at least 99.9 weight-% of the mixture consists of the sofosbuvir, copovidone and the vinyl pyrrolidone-vinyl acetate copolymer;
(ii) processing the mixture obtained from (i) to the solid pharmaceutical composition, wherein the processing comprises
   (ii-1) melt extruding preferably hot melting extruding the mixture obtained from (i) and obtaining an extrudate,
   (ii-2) working the extrudate of (ii-1) and obtaining the solid pharmaceutical composition.

It further contemplated that the solid pharmaceutical composition of the invention is preferably obtained or preferably is obtainable by a process comprising embedding sofosbuvir in a matrix comprising, preferably consisting of the at least one pharmaceutically acceptable matrix compound and one or more pharmaceutically acceptable excipient(s), wherein said embedding comprises melt extruding the at least one pharmaceutically acceptable matrix compound and the one or more pharmaceutically acceptable excipient(s) together with the sofosbuvir, wherein preferably the at least one pharmaceutically acceptable matrix compound is preferably vinyl pyrrolidone-vinyl acetate copolymer, more preferably is copovidone.

It further contemplated that the solid pharmaceutical composition is preferably obtained or preferably is obtainable by a process comprising embedding sofosbuvir in a matrix comprising, preferably consisting of the at least one pharmaceutically acceptable matrix compound and one or more pharmaceutically acceptable excipient(s), by melt extruding the at least one pharmaceutically acceptable matrix compound and one or more pharmaceutically acceptable excipient(s) in solid form together with the sofosbuvir in solid form, preferably by a hot-melt extrusion method.

Preferably according to the invention, the sofosbuvir before (ii), preferably before melt extrusion is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, wherein the crystalline form is preferably crystalline form 1. Form I is prepared according to the process discloses in prepared according to WO 2011/123645 A, Example 10.

It is further contemplated that the solid pharmaceutical composition of the invention as disclosed above is obtainable or obtained by a process comprising
(i′) preparing a mixture of sofosbuvir and the at least one pharmaceutically acceptable matrix compound and at least one solvent;
(ii′) subjecting the mixture of (i′) to drying, preferably by lyophilizing the mixture of (i′) or spray-drying the mixture of (i′), obtaining a dried, preferably lyophilize or spray-dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition.

According to the present invention, the mixture of (i′), depending on the chemical nature of the at least one solvent and the chemical nature of the at least one matrix compound, can be a solution or a dispersion or a suspension.

Amorphous sofosbuvir is preferably formed in step (ii′)

Regarding the at least one solvent, no specific restrictions exist. Preferably, the at least one solvent is selected from the group consisting of C3-C6 ketones such as acetone, C1-C2 halogenated hydrocarbons such as CH₂Cl₂, C1-C4 alcohols such as methanol, C2-C6 ethers, C3-C5 esters such as ethylacetate, a combination of two or more thereof and a combination of one or more thereof with water, more preferably from the group consisting of C1-C4 alcohols such as methanol, C3-C6 ketones such as acetone, and a combination of two or more thereof, wherein more preferably, the at least one solvent comprises, more preferably consists of, and C1-C4 alcohol, preferably methanol, or comprises, more preferably consists of, acetone. Mixture of water and the at least one solvent are also according to the invention.

Regarding the at least one treatment stage from which the amorphous sofosbuvir is obtained, no specific restrictions exist, provided that the amorphous sofosbuvir is obtained. Preferably, the treatment stage comprises subjecting at least a portion of the solution of the sofosbuvir to lyophilization or rapid-drying, preferably to rapid-drying, wherein the rapid-drying preferably comprises at least one atomization process, and is more preferably carried out by spray-drying or spray-granulation, preferably by spray-drying. Prior to the rapid-drying, the solution of the sofosbuvir can be concentrated with respect to the sofosbuvir content, for example by filtration, centrifugation, evaporation, adding sofosbuvir to the solution, or a combination of two or more of these methods.

It is contemplated that the solid pharmaceutical composition of the invention is used in a method for treating hepatitis C in mammals, preferably in a human.

Process for Preparing the Solid Pharmaceutical Composition

Generally, the solid pharmaceutical composition of the present invention can be prepared according to all suitable processes. Preferably, it is prepared by a process comprising comprising
(i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
(ii) processing the mixture obtained from (i) to the solid pharmaceutical composition, wherein preferably the solid pharmaceutical composition.

Preferably, in the process of the invention, the solid pharmaceutical composition according to the invention comprises sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.

Preferably, in the process of the invention, at least 99 weight-% of the sofosbuvir comprised in the solid pharmaceutical composition are present in amorphous form. Preferably, at least 99.5 weight-%, more preferably at least 99.6 weight-%, more preferably at least 99.7 weight-%, more preferably at least 99.8 weight-%, more preferably at least 99.9 weight-% of the sofosbuvir comprised in the solid composition are present in amorphous form. More preferably, at least 99.95 weight-%, more preferably at least 99.99 weight-% of the sofosbuvir comprised in the solid composition are present in amorphous form.

The at least one pharmaceutically acceptable matrix compound and the one or more pharmaceutically acceptable excipient(s) are as disclosed above in the “Solid pharmaceutical composition” section.

As to the at least one pharmaceutically acceptable matrix compound, preferably it is a hydrophilic water-soluble polymer, wherein the hydrophilic water-soluble polymer is preferably selected from the group consisting of polyvinylpyrrolidones (PVP, polyvidone, povidone) such as PVP 40, vinyl pyrrolidone-based copolymers such as vinyl pyrrolidone-vinyl acetate copolymer like copovidone, and other polymers such as polyethylene glycol, polyvinyl acetate and polyvinylcaprolactame-based graft copolymer like Soluplus®.

More preferably, the hydrophilic water-soluble polymer comprises, preferably consists of, a vinyl pyrrolidone-vinyl acetate copolymer, wherein preferably the vinyl pyrrolidone-vinyl acetate copolymer comprises, preferably consists of, copovidone.

According to the invention, preferably from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.

Preferably, in the process of the invention, the solid pharmaceutical composition obtained is an oral dosage, including, but not restricted to, a granule, a capsule, for example a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet or an extrudate.

More preferably, the oral dosage form of the present invention is a tablet.

Step (i)

Any means for preparing the mixture of (i) is suitable according to the present invention.

Preferably according to the invention, in the processes according to the invention, the sofosbuvir of (i) is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, wherein the crystalline form is preferably crystalline form 1.

According to the invention, in the mixture of (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.

Step (ii)

It is preferred that step (ii) is carried out directly. Hence it is contemplated a process wherein
(ii) directly processing the mixture obtained from (i) to the pharmaceutical composition.

Hence, it is preferred that after (i) and before (ii), the mixture obtained from (i) is not subjected to any modification. It is further preferred that the mixture obtained from (i) is processed to the pharmaceutical composition according to (ii) at most 168 h, preferably at most 72 h, more preferably at most 24 h after having been obtained from (i), wherein during this period of time, the mixture is preferably not subjected to stress conditions of 30° C. and a relative humidity of 75%, more preferably stored under ambient conditions.

As to the processing of (ii), the processing comprise, one or more of wet granulation, dry granulation, compression, melting extrusion of the mixture of (i). More preferably the processing of (ii) comprises melting extruding the mixture of (i), more preferably hot melting extruding the mixture of (i).

As to temperature at which the melt extrusion is carried out, the melt extrusion is carried out at a temperature in the range of from 75 to 175° C., preferably in the range of from 90 to 150° C., preferably the melt extrusion is carried out at a temperature of at least 100° C., preferably at least 150° C.

Therefore, the present invention is directed to a process for preparing the solid pharmaceutical composition of the invention, the process comprising
(i) preparing a mixture comprising sofosbuvir, at least one pharmaceutically acceptable matrix compound and one or more pharmaceutically acceptable excipient(s), wherein at least 99 weight-% preferably at least 99.9 weight-% of the mixture consists of the sofosbuvir, the at least one matrix compound and the one or more pharmaceutically acceptable excipient(s);
(ii) processing the mixture obtained from (i) to the solid pharmaceutical composition, wherein the processing comprises
   (ii-1) melt extruding preferably hot melting extruding the mixture obtained from (i) and obtaining an extrudate
   (ii-2) working the extrudate of (ii-1) and obtaining to the solid pharmaceutical composition.

Therefore the present invention is directed to a process for preparing the solid pharmaceutical composition of the invention, the process comprising
(i) preparing a mixture comprising sofosbuvir, a vinyl pyrrolidone-vinyl acetate copolymer, wherein the vinyl pyrrolidone-vinyl acetate copolymer is preferably is copovidone and one or more pharmaceutically acceptable excipient(s), wherein at least 99 weight-%, preferably at least 99.9 weight-% of the mixture consists of the sofosbuvir, copovidone and the vinyl pyrrolidone-vinyl acetate copolymer;
(ii) processing the mixture obtained from (i) to the solid pharmaceutical composition, wherein the processing comprises
   (ii-1) melt extruding, preferably hot melting extruding the mixture obtained from (i) and obtaining an extrudate,
   (ii-2) working the extrudate of (ii-1) and obtaining the solid pharmaceutical composition.

It is contemplated that (i) and (ii) comprise embedding the sofosbuvir in a matrix comprising, preferably consisting of the at least one pharmaceutically acceptable matrix compound and one or more pharmaceutically acceptable excipient(s), wherein said embedding preferably comprises melting extruding the at least one pharmaceutically acceptable matrix compound and the one or more pharmaceutically acceptable excipient(s) together with the sofosbuvir.

It is contemplated that (i) and (ii) comprise embedding sofosbuvir in a matrix comprising, preferably consisting of the at least one pharmaceutically acceptable matrix compound and at least one pharmaceutical acceptable excipient, by melting extruding the at least one pharmaceutically acceptable matrix compound in solid form and the one or more pharmaceutical acceptable excipient(s) in solid form together with the sofosbuvir in solid form, preferably by a hot-melt extrusion method.

It is further contemplated that the (ii), preferably (ii-2) comprises cooling, preferably to a temperature in the range of from 10 to 40° C., preferably in the range of from 20 to 30° C. the extrudate of (ii-1).

It further contemplated a process for preparing the solid pharmaceutical composition of the invention, the process comprising embedding sofosbuvir in a matrix comprising, preferably consisting of the at least one pharmaceutically acceptable matrix compound and one or more pharmaceutically acceptable excipient(s), wherein said embedding comprises melt extruding the at least one pharmaceutically acceptable matrix compound and the one or more pharmaceutically acceptable excipient(s) together with the sofosbuvir, wherein preferably the at least one pharmaceutically acceptable matrix compound is preferably vinyl pyrrolidone-vinyl acetate copolymer, more preferably is copovidone.

Process for Preparing the Solid Pharmaceutical Composition Using a Solvent

Further it is contemplated a process for preparing the solid pharmaceutical composition of the present invention as disclosed above, the process comprising
(i′) preparing a mixture of sofosbuvir and the at least one pharmaceutically acceptable matrix compound and at least one solvent;
(ii′) subjecting the mixture of (i′) to drying, preferably by lyophilizing the mixture of (i′) or spray-drying the mixture of (i′), obtaining a dried, preferably lyophilize or spray-dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition.

Preferably, in the process of the invention, the solid pharmaceutical composition according to the invention comprises sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.

Preferably, in the process of the invention, at least 99 weight-% of the sofosbuvir comprised in the solid pharmaceutical composition are present in amorphous form. Preferably, at least 99.5 weight-%, more preferably at least 99.6 weight-%, more preferably at least 99.7 weight-%, more preferably at least 99.8 weight-%, more preferably at least 99.9 weight-% of the sofosbuvir comprised in the solid composition are present in amorphous form. More preferably, at least 99.95 weight-%, more preferably at least 99.99 weight-% of the sofosbuvir comprised in the solid composition are present in amorphous form.

According to the invention, in the mixture of (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1. The term “the at least one matrix compound” as used in this context of the present invention relates to the sum of all matrix compounds employed.

Regarding the preferred matrix compounds and the one or more pharmaceutically acceptable excipients, reference can be made to respective description above, in the section “Solid pharmaceutical composition”.

Regarding the at least one pharmaceutically acceptable matrix compound it is preferably a silicon-based inorganic adsorbents they include as defined above, preferably are, one or more of silica and silicates. Examples of such silica include, but are not restricted to fumed silica, precipitated silica, gel silica, colloidal silica, such as Syloid® AL-1 FP and Syloid® 72FP silica and Syloid® 244FP silica.

It is further preferred that the at least one matrix compound has a pH in the range of from 5.0 to 9.0, preferably in the range of from 6.5 to 8.5, more preferably in the range of from 7.0 to 8.0.

Preferably, in this process of the invention at least 99.5 weight-%, preferably at least 99.9 weight-% of the solid pharmaceutical composition consists of the sofosbuvir the at least one matrix compound and one or more pharmaceutically acceptable excipient(s).

According to the invention, preferably from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.

Preferably, in the process of the invention, the solid pharmaceutical composition obtained is an oral dosage, including, but not restricted to, a granule, a capsule, for example a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet or an extrudate.

More preferably, the oral dosage form of the present invention is a tablet.

Therefore, it is contemplated a process for preparing the solid pharmaceutical composition of the present invention, the process comprising
(i′) preparing a mixture of sofosbuvir and the at least one pharmaceutically acceptable matrix compound and at least one solvent;
(ii′) subjecting the mixture of (i′) to drying, preferably by lyophilizing the mixture of (i′) or spray-drying the mixture of (i′), obtaining a dried, preferably lyophilize or spray-dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition,
   wherein from 30 to 45 weight-%, more preferably from 30 to 40 weight-%, more preferably 40 weight-% of the solid pharmaceutical composition consists of sofosbuvir based on the total weight of the solid pharmaceutical composition and wherein at least 99.5 weight-%, preferably at least 99.9 weight-% of the sofosbuvir comprised in the solid composition are present in amorphous form and wherein at least 99.5 weight-%, preferably at least 99.9 weight-% of the solid pharmaceutical composition consists of the sofosbuvir, the at least one matrix compound and one or more pharmaceutically acceptable excipient(s).

Therefore, it is contemplated a process for preparing the solid pharmaceutical composition of the present invention, the process comprising
(i′) preparing a mixture of sofosbuvir and the at least one pharmaceutically acceptable matrix compound and at least one solvent;
(ii′) subjecting the mixture of (i′) to drying, preferably by lyophilizing the mixture of (i′) or spray-drying the mixture of (i′), obtaining a dried, preferably lyophilize or spray-dried mixture-1,
(iii′) mixing the mixture-1 of (ii′) with the one or more pharmaceutically acceptable excipient(s), obtaining a mixture-2
(iv′) processing the mixture-2 of (iii′) to the solid pharmaceutical composition,
   wherein from 20 to 45 weight-%, more preferably from 20 to 40 weight-%, more preferably from 30 to 40 weight-%, more preferably 30 weight-% of the solid pharmaceutical composition consists of sofosbuvir based on the total weight of the solid pharmaceutical composition, wherein at least 99.5 weight-%, preferably at least 99.9 weight-% of the sofosbuvir comprised in the solid composition are present in amorphous form, wherein at least one pharmaceutically acceptable matrix compound is a silicon-based inorganic adsorbents, wherein the silicon-based inorganic adsorbents is preferably one or more of silica and silicates and wherein at least 99.5 weight-%, preferably at least 99.9 weight-% of the solid pharmaceutical composition consists of the sofosbuvir, the at least one matrix compound and one or more pharmaceutically acceptable excipient(s).

Step (i′)

Any method for preparing the mixture of step (i′) is suitable according to the present invention. For example the mixture of (i′) can be prepared by dissolving the sofosbuvir in at least one solvent and subsequently adding the pharmaceutically acceptable matrix compound.

According to the process of the present invention, the mixture of (i′), depending on the chemical nature of the at least one solvent and the chemical nature of the at least one matrix compound, can be a solution or a dispersion or a suspension.

Generally, no specific restrictions exist which solvent or which mixture or combination of solvents is used, provided that the sofosbuvir can be essentially dissolved therein. The term “essentially dissolved” as used in this context of the present invention relates to a process wherein at least 99 weight-%, preferably at least 99.9 weight-%, more preferably at least 99.99 weight-% of the sofosbuvir is dissolved.

Regarding the at least one solvent, no specific restrictions exist. Preferably, the at least one solvent is selected from the group consisting of C3-C6 ketones such as acetone, C1-C2 halogenated hydrocarbons such as CH₂Cl₂, C1-C4 alcohols such as methanol, C2-C6 ethers, C3-C5 esters such as ethylacetate, and a combination of two or more thereof, more preferably from the group consisting of C1-C4 alcohols such as methanol, C3-C6 ketones such as acetone, and a combination of two or more thereof, wherein more preferably, the at least one solvent comprises, more preferably consists of, and C1-C4 alcohol, preferably methanol, or comprises, more preferably consists of, acetone. Mixture of water and the at least one solvent are also according to the invention.

According to the invention, in the mixture of (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.

Step (ii′)

Generally, no specific restrictions exist how said drying is carried. Conceivable drying methods include, but are not restricted to, direct drying, such as batch drying in a suitable oven or continuous drying or spray-drying or spray-granulation, for example in a band drying apparatus, or filtration or centrifugation; indirect drying, such as drum drying or vacuum drying; and freeze drying such as lyophilization. A combination of two or more different drying methods can be applied. Preferably, the mixture comprising the sofosbuvir, the at least one pharmaceutically acceptable matrix compound and the at least one solvent, wherein preferably the mixture is a suspension, is subjected to direct drying, preferably spray-drying, or freeze drying, preferably lyophilization. Therefore, the present invention also relates to the process as described above, wherein the process comprises subjecting the mixture, preferably the suspension, comprising the sofosbuvir, the pharmaceutically acceptable matrix compound and the at least one solvent to drying by lyophilizing the mixture or spray-drying the mixture.

It is preferred that in the process of the invention (i′) and (ii′) comprise embedding sofosbuvir in a matrix comprising, preferably consisting of the at least one pharmaceutically acceptable matrix compound, wherein said embedding comprises preparing a mixture, wherein preferably the mixture is a suspension, of the sofosbuvir, the at least one pharmaceutically acceptable matrix compound and at least one solvent and subjecting said mixture to drying, preferably by lyophilizing the mixture or spray-drying the mixture, wherein preferably the mixture is a suspension.

Step (iii′)

In step (iii′), the mixing of the mixture of (ii′) with the one or more pharmaceutically acceptable excipient(s) is carried out to obtain a mixture-1. The one or more pharmaceutically acceptable excipient(s) are as defined above.

Step (iv′)

In step (iv′) the processing the mixture to the solid pharmaceutical composition, is carried out. It is preferred that the processing of (iv′) comprise, preferably is one or more of wet granulation, dry granulation, compression, melting extrusion of the mixture of (iii′). More preferably (iv′) comprises, preferably consists in melting extruding the mixture of (iii′), more preferably hot melting extruding the mixture of (iii′).

Pharmaceutical Composition Comprising Amorphous Sofosbuvir without Matrix Compounds

In an aspect the present invention is directed to a solid pharmaceutical composition comprising the amorphous sofosbuvir which does not comprise the above mentioned matrix compounds. Any method for preparing amorphous sofosbuvir is contemplated according to the invention. Preferably the amorphous sofosbuvir is prepared by rapid dry, preferably spray dry and then directly formulated with one or more pharmaceutically acceptable excipient(s). It has been seen that amorphous sofosbuvir obtained by rapid dry, preferably spray dry is stable in the solid pharmaceutical formulation. Advantageously by mean of the rapid dry and spray dry techniques a better control of the particle size of amorphous sofosbuvir is obtained. Further, the solvent is easily removed and a lower residue of solvent is present in the spray dried amorphous sofosbuvir.

In an aspect the present invention is further directed to a solid pharmaceutical composition wherein the solid pharmaceutical composition comprises sofosbuvir according to formula (I)

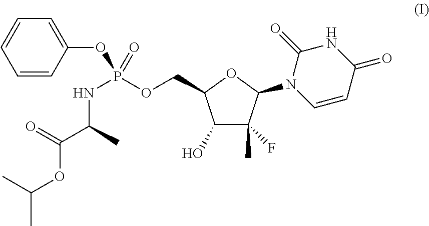 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir and the one or more pharmaceutically acceptable excipient(s).

Preferably, according to the invention, at least 99.5 weight-%, more preferably at least 99.6 weight-%, more preferably at least 99.7 weight-%, more preferably at least 99.8 weight-%, more preferably at least 99.9 weight-% of the sofosbuvir comprised in the solid composition are present in amorphous form. More preferably, at least 99.95 weight-%, more preferably at least 99.99 weight-% of the sofosbuvir comprised in the solid composition are present in amorphous form.

According to the invention, the solid pharmaceutical composition comprises sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition. The remaining of the composition, up to 100 weight-% of the composition comprises, preferably consists of the one or more pharmaceutically acceptable excipients.

Preferably from 20 to 45 weight-%, more preferably from 20 to 40 weight-%, more preferably from 30 to 40 weight-%, more preferably 30 weight-% of the composition consists of sofosbuvir based on the total weight of the solid pharmaceutical composition. The remaining of the composition up to 100 weight-% of the composition comprises, preferably consists of the one or more pharmaceutically acceptable excipients.

Preferably, 30 or 35 or 40 or 45 weight-% of the solid pharmaceutical composition consists of sofosbuvir based on the total weight of the solid pharmaceutical composition. The remaining of the composition up to 100 weight-% comprises, preferably consists of the one or more pharmaceutically acceptable excipients.

Hence, according to the present invention, at least 99.5 weight-%, preferably at least 99.9 weight-% of the solid pharmaceutical composition consists of the sofosbuvir and one or more pharmaceutically acceptable excipient(s).

According to the invention, the solid pharmaceutical composition being is oral dosage selected from the group consisting of a granule, a capsule such as a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet and an extrudate, preferably the solid pharmaceutical composition is a tablet or a coated tablet.

It is contemplated that the solid pharmaceutical composition according to this aspect of the invention comprises further comprising, in addition to the sofosbuvir, one or more further HCV agents including one or more of ledipasvir according to formula (II)

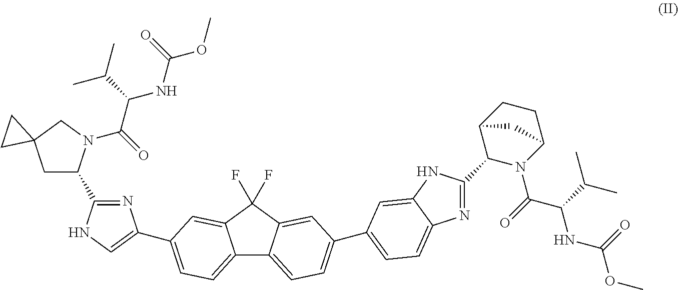 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

and daclatasvir of formula (III)

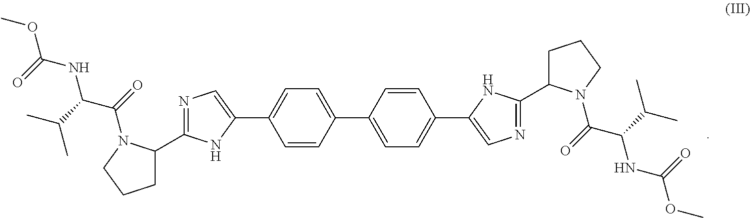

Preferably, the solid pharmaceutical composition consists of sofosbuvir, and the one or more pharmaceutically acceptable excipient(s) and optionally the one or more further HCV agents.

The solid pharmaceutical composition according to this aspect of the invention comprises one or more pharmaceutically acceptable excipients. These excipients as defined above in relation to the aspect of the invention wherein a matrix compound is present in the solid pharmaceutical composition. It is preferred that the excipients are not the pharmaceutically acceptable matrix compound as disclosed above. For example it is preferred that the one or more excipient is not povidone such as PLASDONE™ S-630 or polyvinylpyrrolidine K-30.

Preferably the one or more pharmaceutically acceptable excipient(s) comprise, preferably are, a combination of at least one a diluent, at least one disintegrant, at least one glidant, at least one lubricant and optionally at least one coating agent.

Therefore, the solid pharmaceutical composition according to this aspect of the invention, comprises:
from 25 to 35 weight-%, preferably from 25 to 30 weight-% diluent,
from 25 to 40 weight-%, preferably from 25 to 30 weight-% disintegrant,
from 0.5 to 7 weight-%, preferably from 0.8 to 5 weight-% glidant,
from 1 to 6 weight-%, preferably from 1.4 to 2 weight-% lubricant,
   in each based on the total weight of the solid pharmaceutical composition.

Therefore, the solid pharmaceutical composition of this aspect of the invention comprises
from 15 to 45 weight-%, preferably from 25 to 40 weight-%, more preferably
from 30 to 40 weight-% sofosbuvir,
from 25 to 35 weight-%, preferably from 25 to 30 weight-% diluent,
from 25 to 40 weight-% preferably from 25 to 30 weight-% disintegrant,
from 0.5 to 7 weight-% preferably from 0.8 to 5 weight-% glidant,
from 1 to 6 weight-% preferably from 1.4 to 2 weight-% lubricant, and optionally
from 1 to 3.5 preferably from 2 to 3 weight-% coating agent,
   in each based on the total weight of the solid pharmaceutical composition.

According to this aspect of the invention, the one or more pharmaceutically acceptable excipient(s) comprise, preferably are, a combination of mannitol, microcrystalline cellulose, croscarmellose sodium, silica dioxide, magnesium stearate and optionally a coating agent.

Therefore, the solid pharmaceutical composition of this aspect of the invention comprises
from 25 to 35 weight-%, preferably from 25 to 30 weight-% mannitol,
from 25 to 34 weight-%, preferably from 25 to 30-% weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
from 1 to 6 weight-%, preferably from 1.4 to 2 weight-% magnesium stearate, and
optionally from 1.5 to 3.5 weight-% coating agent,
in each based on the total weight of the solid pharmaceutical composition.

Therefore, the solid pharmaceutical composition of this aspect of the invention comprises
from 25 to 45 weight-%, preferably from 30 to 40 weight-% sofosbuvir,
from 25 to 35 weight-%, preferably from 25 to 30 weight-% mannitol,
from 25 to 36 weight-%, preferably from 25 to 30-% weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
from 1 to 6 weight-%, preferably from 1.4 to 2 weight-% magnesium stearate, and
optionally from 1.5 to 3.5 weight-% coating agent, preferably Opadry II.
   in each based on the total weight of the solid pharmaceutical composition.

Therefore, the solid pharmaceutical composition of this aspect of the invention comprises
from 30 to 40 weight-% sofosbuvir,
from 26 to 29 weight-% mannitol,
from 26 to 28 weight-% microcrystalline cellulose,
from 3.5 to 5.5 weight-% croscarmellose sodium,
from 0.8 to 4 weight-% colloidal silica,
optionally from 1.4 to 2 weight-% magnesium stearate,
optionally from 2.5 to 3.5 weight-% coating agent such as a film forming agent, preferably Opadry II
   in each based on the total weight of the solid pharmaceutical composition, wherein the individual contents add up to 100%.

Preferably, according to this aspect of the invention, the solid pharmaceutical composition is a tablet preferably selected from the group consisting of an uncoated tablet, a coated tablet, an effervescent tablet, a soluble tablet, a dispersible tablet, an orodispersible tablet, a tablet for use in the mouth, a chewable tablet preferably the solid pharmaceutical composition is a tablet or a coated tablet

Preferably, the oral dosage form, preferably the tablet or the coated tablet has a weight in the range of from 900 mg to 1300 mg.

It is preferred that according to this aspect of the invention, the amorphous sofosbuvir is obtainable or obtained by a process comprising sofosbuvir by rapid drying, preferably by spray drying. It is preferred that the amorphous sofosbuvir is obtained by spray drying a solution comprising sofosbuvir and one or more solvents.

It is preferred hence that that the solid pharmaceutical compositions according to this aspect of the invention are obtainable or obtained by a process comprising
(i) providing amorphous sofosbuvir.
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

It is further preferred hence that that the solid pharmaceutical compositions according to this aspect of the invention are obtainable or obtained by a process comprising
(i) preparing amorphous sofosbuvir by rapid drying, preferably by spray drying a solution comprising sofosbuvir and one or more solvents;
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

It is preferred that step (iii) consists of
(iii) directly processing the mixture obtained from (ii) to the pharmaceutical composition.

It is further preferred that after (ii) and before (iii), the mixture obtained from (ii) is not subjected to any modification.

It is further preferred that the mixture obtained from (ii) is processed to the pharmaceutical composition according to (iii) at most 168 h, preferably at most 72 h, more preferably at most 24 h after having been obtained from (ii), wherein during this period of time, the mixture is preferably not subjected to stress conditions of 30° C. and a relative humidity of 75%, more preferably stored under ambient conditions.

It is further preferred that the processing of (iii) comprises one or more of wet granulation, dry granulation, compression, melting extrusion of the mixture of (ii). More preferably, the processing of (iii) comprises direct compression of the mixture of (ii). Alternatively, the processing of (iii) comprises a dry granulation step.

When a granulation process is carried out first a intragranular composition comprising the amorphous sofosbuvir and first portion of one or more pharmaceutically acceptable excipients is prepared and granulated to obtain an intragranular composition. The intragranular composition is then mixed with the second portion (extragranular composition) of the one or more pharmaceutically acceptable excipients. The mixture of the intragranular and extragranular compositions is then preferably compressed to form a tablet. In this case the mixture of (ii) is the intragranular composition. In step (iii), the intragranular composition is processed with the extragranular composition to the solid pharmaceutical composition of the invention.

It is preferred that (iii) further comprises coating the solid pharmaceutical composition.

It is preferred that (i) comprises
(i′) preparing a solution of sofosbuvir and one or more solvents.

It is preferred that the one or more solvents is selected from the group consisting of an organic solvent, and a combination of two or more thereof. According to the invention, the organic solvent is preferably selected from the group consisting of a C1-C2 halogenated hydrocarbon such as CH₂Cl₂, a C1-C4 alcohol, such as a C1 alcohol such as methanol, a C2 alcohol such as ethanol, a C3 alcohol such as propanol, or a C4 alcohol such as butanol; a C3-C6 ketone such as a C3 ketone such as acetone, a C4 ketone, a C5 ketone, or a C6 ketone, a C2-C6 ether such as C2 ether, a C3 ether, a C4 ether, a C5 ether, or C6 ether; a C3-C5 ester such as a C3 ester, a C4 ester, or a C5 ester such as ethylacetate, a combination of two or more thereof and a combination of one or more thereof with water.

According to the present invention, it is preferred that the rapid draying is spray drying.

Therefore, the solid pharmaceutical composition of this aspect of the invention comprises
from 25 to 35 weight-%, preferably from 25 to 30 weight-% mannitol,
from 25 to 34 weight-%, preferably from 25 to 30-% weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
from 1 to 6 weight-%, preferably from 1.4 to 2 weight-% magnesium stearate, and
optionally from 1.5 to 3.5 weight-% coating agent,
   in each based on the total weight of the solid pharmaceutical composition wherein the composition is obtainable or is obtained by a process comprising
(i) preparing amorphous sofosbuvir
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

Amorphous sofosbuvir of (i) can be prepared by rapid drying, preferably by spray drying a solution comprising sofosbuvir and one or more solvents;

Therefore, the solid pharmaceutical composition of this aspect of the invention comprises
from 25 to 45 weight-%, preferably from 30 to 40 weight-% sofosbuvir,
from 25 to 35 weight-%, preferably from 25 to 30 weight-% mannitol,
from 25 to 36 weight-%, preferably from 25 to 30-% weight-% microcrystalline cellulose,
from 2 to 6 weight-%, preferably from 2.5 to 5.5 weight-% croscarmellose sodium,
from 0.5 to 7 weight-%, preferably from 0.8 to 5 weight-% silica dioxide,
from 1 to 6 weight-%, preferably from 1.4 to 2 weight-% magnesium stearate, and
optionally from 1.5 to 3.5 weight-% coating agent such as a film forming agent, preferably Opadry II.
   in each based on the total weight of the solid pharmaceutical composition wherein the composition is obtainable or is obtained by a process comprising
(i) preparing amorphous sofosbuvir;
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

Amorphous sofosbuvir of (i) can be prepared by rapid drying, preferably by spray drying a solution comprising sofosbuvir and one or more solvents.

Therefore, the solid pharmaceutical composition of this aspect of the invention comprises
from 30 to 40 weight-% sofosbuvir,
from 26 to 29 weight-% mannitol,
from 26 to 28 weight-% microcrystalline cellulose,
from 3.5 to 5.5 weight-% croscarmellose sodium,
from 0.8 to 4 weight-% colloidal silica,
optionally from 1.4 to 2 weight-% magnesium stearate,
optionally from 2.5 to 3.5 weight-% coating agent, preferably Opadry II in each based on the total weight of the solid pharmaceutical composition, wherein the individual contents add up to 100%, wherein the composition is obtainable or is obtained by a process comprising
(i) preparing amorphous sofosbuvir;
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

Amorphous sofosbuvir can be prepared by rapid drying, preferably by spray drying a solution comprising sofosbuvir and one or more solvents.

Therefore, the solid pharmaceutical composition of this aspect of the invention comprises
from 30 to 40 weight-% sofosbuvir,
from 26 to 29 weight-% mannitol,
from 26 to 28 weight-% microcrystalline cellulose,
from 3.5 to 5.5 weight-% croscarmellose sodium,
from 0.8 to 4 weight-% colloidal silica,
from 1.4 to 2 weight-% magnesium stearate,
optionally from 2.5 to 3.5 weight-% coating agent, preferably Opadry II
   in each based on the total weight of the solid pharmaceutical composition, wherein the individual contents add up to 100%, wherein the composition is obtainable or is obtained by a process comprising
(i) preparing amorphous sofosbuvir
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition,
   wherein the mixture of (ii) is an intragranular composition and in (iii), the intragranular composition of (ii) is processed with an extragranular composition to the solid pharmaceutical composition of the invention and wherein (iii) optionally comprises a coating step.

Amorphous sofosbuvir of (i) can be prepared by rapid drying, preferably by spray drying a solution comprising sofosbuvir and one or more solvents;

Hence, the present invention is further directed to a process for preparing the solid pharmaceutical composition according to this aspect of the invention as disclose above.

### Uses

The solid pharmaceutical composition, preferably the oral dosage form, more preferably the tablet of the present invention as disclosed above in all the aspects is preferably used in a method for treating hepatitis C in mammals, preferably in a human. Therefore, the present invention also relates to a solid pharmaceutical composition as described above, for use in a method for treating hepatitis C in a human. Further, the present invention relates to the use of a solid pharmaceutical composition as described above for treating hepatitis C in a human. Further, the present invention relates to the use of a solid pharmaceutical composition as described above for the preparation of a medicament for treating hepatitis C in a human. Further, the present invention relates to a method for treating hepatitis C comprising administering a solid a pharmaceutical composition as described above to a human patient in need thereof.

According to a further aspect, the present invention is illustrated by the following embodiments and combinations of embodiments resulting from the given dependencies and back-references:
1. A solid pharmaceutical composition wherein the solid pharmaceutical composition comprises sofosbuvir according to formula (I)

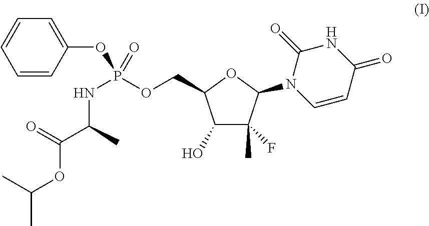 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s).
2. The solid pharmaceutical composition of embodiment 1, wherein the solid pharmaceutical composition comprises the sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.
3. The solid pharmaceutical composition of embodiment 1 or 2, wherein the at least one matrix compound is selected from the group consisting of hydrophilic polymers, silicon-based inorganic adsorbents and a combination of two or more thereof.
4. The solid pharmaceutical composition of embodiment 3, wherein the silicon-based inorganic adsorbents include, preferably are, one or more of silica and silicates.
5. The solid pharmaceutical composition of embodiment 3 or 4, wherein the at least one silicon-based inorganic adsorbent has an oil absorbance in the range of from 1.0 to 5.0 ml/g, preferably in the range of from 1.5 to 4.0 ml/g.
6. The solid pharmaceutical composition of any one of embodiments 3 to 5, wherein the at least one silicon-based inorganic adsorbent has a bulk density in the range of from 10 to 600 g/ml, preferably in the range of from 30 to 500 g/ml, more preferably in the range of from 50 to 300 g/ml, more preferably in the range of from 50 to 200 g/ml.
7. The solid pharmaceutical composition of any one of embodiments 5 to 7, wherein the at least one silicon-based inorganic adsorbent is selected from the group consisting of silica, silicates, and a combination of two or more thereof, wherein the silica is preferably selected from the group consisting of fumed silica, precipitated silica, gel silica, colloidal silica, and a combination of two or more thereof, and wherein the silicates are preferably aluminosilicates preferably comprising at least one alkali metal element and/or at least one alkaline earth metal element, more preferably at least one alkaline earth metal element, more preferably magnesium, wherein more preferably, at least 90 weight-%, more preferably at least 95 weight-%, more preferably at least 99 weight-% of the at least one silicon-based inorganic adsorbent are present in amorphous form.
8. The solid pharmaceutical composition of embodiment 3, wherein the hydrophilic polymers include, preferably are, one or more of polysaccharides, preferably cellulose derivatives such as hydroxyalkylalkylcelullose, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.
9. The solid pharmaceutical composition of embodiment 3 or 8, wherein the at least one matrix compound is selected from the group consisting of hydrophilic water-soluble polymers and a combination of two or more thereof.
10. The solid pharmaceutical composition of embodiment 9, wherein the at least one hydrophilic water-soluble polymer has a solubility in water of at least 10 g/l, preferably of at least 20 g/l, more preferably of at least 30 g/l, in each case at 23° C. at atmospheric pressure.
11. The solid pharmaceutical composition of embodiment 9 or 10, wherein the at least one hydrophilic water-soluble polymer comprises, preferably consists of, at least one vinyl pyrrolidone-vinyl acetate copolymer.
12. The solid pharmaceutical composition of any one of embodiments 9 to 11, wherein the at least one hydrophilic water-soluble polymer comprises, preferably consists of, copovidone.
13. The solid pharmaceutical composition of any one of embodiments 9 to 12, wherein the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.
14. The solid pharmaceutical composition of embodiment 3 or 8, wherein the at least one hydrophilic water-soluble polymer comprises, preferably consists of a cellulose derivative selected from the group consisting of hydroxyalkylalkylcelluloses and a mixture of two or more thereof, the at least one hydrophilic water-soluble polymer preferably comprising, more preferably consisting of, hydroxypropylmethylcellulose (HPMC).
15. The solid pharmaceutical composition of embodiment 14, wherein the cellulose derivative has a degree of substitution (DS) in the range of from 0.3 to 2.8, preferably in the range of from 0.6 to 2.5, more preferably in the range of from 1.0 to 2.3, more preferably in the range of from 1.3 to 2.0.
16. The solid pharmaceutical composition of embodiment 14 or 15, wherein the weight average molecular weight (M_{w}) of the cellulose derivative is in the range of from 7 to 225 kDa, preferably in the range of from 7 to 100 kDa, more preferably in the range of from 7 to 30 kDa.
17. The solid pharmaceutical composition of any one of embodiments 1 to 16, wherein at least 99.5 weight-%, preferably at least 99.9 weight-% of the sofosbuvir comprised in the composition are present in amorphous form. 18. The solid pharmaceutical composition of any one of embodiments 1 to 17, wherein at least 99.5 weight-%, preferably at least 99.9 weight-% of the solid pharmaceutical composition consists of the sofosbuvir, the at least one matrix compound and one or more pharmaceutically acceptable excipient(s).
19. The solid pharmaceutical composition of any one of embodiments 1 to 18, wherein from 30 to 60 weight-% or from 30 to 50 weight-% or from 35 to 50 weight-% of the solid pharmaceutical composition consists of sofosbuvir based on the total weight of the solid pharmaceutical composition.
20. The solid pharmaceutical composition of any one of embodiments 1 to 19, wherein from 30 to 45 weight-%, preferably from 30 to 40 weight-% of the solid pharmaceutical composition consists of sofosbuvir based on the total weight of the solid pharmaceutical composition.
21. The solid pharmaceutical composition of any one of embodiments 1 to 20, wherein from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.
22. The solid pharmaceutical composition of any one of embodiments 1 to 21, wherein the solid pharmaceutical composition comprises less than 0.1 weight-%, preferably less than 0.01 weight-%, more preferably less than 0.001 weight-% of a surfactant.
23. The solid pharmaceutical composition of any one of embodiments 1 to 22, wherein the solid pharmaceutical composition is a solid dispersion.
24. The solid pharmaceutical composition of any one of embodiments 1 to 23, being an oral dosage form, preferably a tablet.
25. The solid pharmaceutical composition of any one of embodiments 1 to 24, further comprising, in addition to the sofosbuvir, one or more further HCV agents including one or more of ledipasvir according to formula (II)

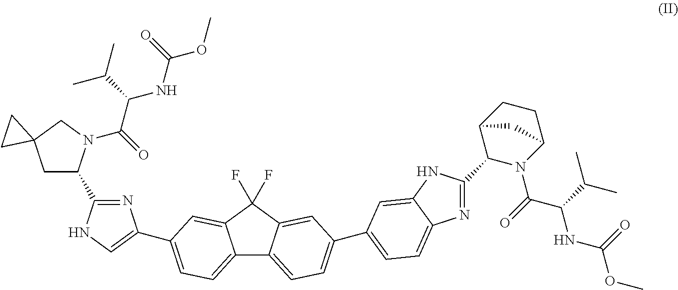 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

and daclatasvir of formula (III)

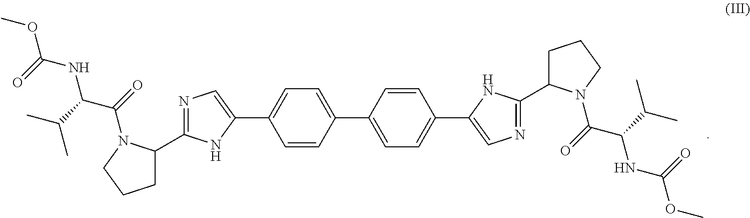

26. The solid pharmaceutical composition of any one of embodiments 1 to 25, wherein the solid pharmaceutical composition consists of sofosbuvir, the at least one pharmaceutically acceptable matrix compound, the one or more pharmaceutically acceptable excipient(s) and optionally the one or more further HCV agents.
27. The solid pharmaceutical composition of embodiment 26, wherein at least one of the one or more pharmaceutically acceptable excipient(s) is different from the at least one pharmaceutically acceptable matrix compound comprised in the solid pharmaceutical composition.
28. The solid pharmaceutical composition of embodiment 27, wherein the at least one of the one or more pharmaceutically acceptable excipient(s) is not a pharmaceutically acceptable matrix compound as defined in any one of embodiments 3 to 16.
29. The solid pharmaceutical composition of any one of embodiments 1 to 28, wherein the one or more pharmaceutically acceptable excipient(s) comprise one or more of at least one of a diluent, at least one disintegrant, at least one glidant, optionally at least one lubricant, and combinations of two or more thereof. 30. The solid pharmaceutical composition of embodiment 29, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
31. The solid pharmaceutical composition of embodiment 30, wherein the at least one diluent is mannitol.
32. The solid pharmaceutical composition of embodiment 29 or 30, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
33. The solid pharmaceutical composition of embodiment 31, wherein the at least one disintegrant is selected from croscarmellose sodium, such as Ac-Di-Sol® croscarmellose sodium.
34. The solid pharmaceutical composition of any one of embodiments 29 to 33, wherein the at least one glidant includes one or more of colloidal silicon dioxide, talc, starch, starch derivatives.
35. The solid pharmaceutical composition of embodiment 34, wherein the at least one glidant is colloidal silicon dioxide.
36. The solid pharmaceutical composition of any one of embodiments 29 to 35, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
37. The solid pharmaceutical composition of embodiment 36, wherein the at least one glidant is magnesium stearate.
38. The solid pharmaceutical composition of any one of embodiments 29 to 37, wherein the one or more pharmaceutically acceptable excipient(s) comprise, preferably are, a combination of at least one a diluent, at least one disintegrant, at least one glidant, and optionally at least one lubricant.
39. The solid pharmaceutical composition of any one of embodiments 29 to 38, comprising
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
40. The solid pharmaceutical composition of any one of embodiments 29 to 39, comprising
   from 30 to 45 weight-%, preferably from 30 to 40 weight-% sofosbuvir,
   from 5 to 15 weight-%, preferably from 7 to 13 weight-% of the pharmaceutically acceptable matrix compound,
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
41. The solid pharmaceutical composition of any one of embodiments 29 to 42, wherein the one or more pharmaceutically acceptable excipient(s) comprise, preferably are, a combination of mannitol, microcrystalline cellulose, croscarmellose sodium, silica dioxide, magnesium stearate.
42. The solid pharmaceutical composition of embodiment 41, comprising
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
43. The solid pharmaceutical composition of embodiment 42, comprising
   from 30 to 45 weight-%, preferably from 30 to 40 weight-% sofosbuvir,
   from 5 to 15 weight-%, preferably from 7 to 13 weight-% copovidone,
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
44. The solid pharmaceutical composition of embodiment 41 or 42, comprising
   from 30 to 40 weight-% sofosbuvir,
   from 7 to 13 weight-% copovidone,
   from 22 to 26 weight-% mannitol,
   from 24 to 28 weight-% microcrystalline cellulose,
   from 2.5 to 5.5 weight-% croscarmellose sodium,
   from 0.8 to 5 weight-% colloidal silica,
   optionally from 1.4 to 2 weight-% magnesium stearate,
   in each based on the total weight of the solid pharmaceutical composition, wherein the individual contents add up to 100%.
45. The solid pharmaceutical composition of any one of embodiments 1 to 44, which is a tablet.
46. The solid pharmaceutical composition of embodiment 45, wherein the tablet is a coated tablet.
47. The solid pharmaceutical composition of embodiments 45 or 46, wherein the tablet has a weight in the range of from 900 mg to 2300 mg, preferably from 1000 mg to 2000 mg.
48. The solid pharmaceutical composition of any one of embodiments 1 to 47, obtainable or obtained by a process comprising
   (i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
   (ii) processing the mixture obtained from (i) to the solid pharmaceutical composition, wherein preferably the solid pharmaceutical composition is a tablet.
49. The solid pharmaceutical composition of embodiment 48, said process comprising
   (ii) directly processing the mixture obtained from (i) to the pharmaceutical composition.
50. The solid pharmaceutical composition of embodiment 48 or 49, wherein after (i) and before (ii), the mixture obtained from (i) is not subjected to any modification.
51. The solid pharmaceutical composition of any one of embodiments 48 to 50, wherein the mixture obtained from (i) is processed to the pharmaceutical composition according to (ii) at most 168 h, preferably at most 72 h, more preferably at most 24 h after having been obtained from (i), wherein during this period of time, the mixture is preferably not subjected to stress conditions of 30° C. and a relative humidity of 75%, more preferably stored under ambient conditions.
52. The solid pharmaceutical composition of any one of embodiments 48 to 51, wherein (ii) comprise one or more of wet granulation, dry granulation, compression, melting extrusion of the mixture of (i).
53. The solid pharmaceutical composition of any one of embodiments 48 to 52, wherein (ii) comprise melting extruding the mixture of (i), preferably hot melting extruding the mixture of (i).
54. The solid pharmaceutical composition of any one of embodiments 1 to 53, wherein the solid pharmaceutical composition is prepared by a process comprising embedding sofosbuvir in a matrix comprising, preferably consisting of the at least one pharmaceutically acceptable matrix compound and one or more pharmaceutically acceptable excipient(s), wherein said embedding comprises melt extruding the at least one pharmaceutically acceptable matrix compound and the one or more pharmaceutically acceptable excipient(s) together with the sofosbuvir.
55. The solid pharmaceutical composition of embodiment 1 to 54, wherein the solid pharmaceutical composition is prepared by a process comprising embedding sofosbuvir in a matrix comprising, preferably consisting of the at least one pharmaceutically acceptable matrix compound and one or more pharmaceutically acceptable excipient(s), by melt extruding the at least one pharmaceutically acceptable matrix compound and one or more pharmaceutically acceptable excipient(s) in solid form together with the sofosbuvir in solid form, preferably by a hot-melt extrusion method.
56. The solid pharmaceutical composition of any one of embodiments 52 to 55, wherein the sofosbuvir before melt extrusion is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, preferably crystalline form 1.
57. The solid pharmaceutical composition of any one of embodiments 1 to 56 wherein at least 99 weight-%, preferably at least 99.5 weight-%, more preferably at least 99.9 weight-% of the sofosbuvir comprised in the solid pharmaceutical composition are present in amorphous form.
58. The solid pharmaceutical composition of any one of embodiments 53 to 77, wherein the melt extrusion is carried out at a temperature of at least 100° C., preferably at least 150° C.
59. The solid pharmaceutical composition of any one of embodiments 1 to 58, obtainable or obtained by a process comprising
   (i′) preparing a mixture of sofosbuvir and the at least one pharmaceutically acceptable matrix compound and at least one solvent;
   (ii′) subjecting the mixture of (i′) to drying, preferably by lyophilizing the mixture of (i′) or spray-drying the mixture of (i′), obtaining a dried, preferably lyophilize or spray-dried mixture-1;
   (iii′) mixing the mixture-1 of (ii′) with the one or more pharmaceutically acceptable excipient(s), obtaining a mixture-2;
   (iv′) processing the mixture-2 of (iii′) to the solid pharmaceutical composition.
60. The solid pharmaceutical composition of embodiment 59, wherein the mixture of (i′), is a solution or a dispersion or a suspension.
61. The solid pharmaceutical composition of any one of embodiments 1 to 60, wherein the solid pharmaceutical composition is obtained or obtainable by a process comprising embedding sofosbuvir in a matrix comprising, preferably consisting of the at least one pharmaceutically acceptable matrix compound, wherein said embedding comprises preparing a mixture of the sofosbuvir, the at least one pharmaceutically acceptable matrix compound and at least one solvent preferably selected from the group consisting of an organic solvent, and a combination of two or more thereof, more preferably selected from the group consisting of a C1-C2 halogenated hydrocarbon, a C1-C4 alcohol, a C3-C6 ketone, a C2-C6 ether, a C3-C5 ester, a combination of two or more thereof and a combination of one or more thereof with water, and subjecting said mixture to drying, preferably by lyophilizing the solution or spray-drying the mixture.
62. The solid pharmaceutical composition of embodiment 61, wherein the process further comprises mixing to the dried, preferably lyophilize or spray-dried solution with the one or more pharmaceutically acceptable excipient(s) to the dried, preferably lyophilized or spray-dried solution, obtaining a mixture and processing said mixture to the solid pharmaceutical composition.
63. The solid pharmaceutical composition of any one of embodiments 59 to 62, wherein the organic solvent is selected from the group consisting of C3-C6 ketones such as acetone, C1-C2 halogenated hydrocarbons such as CH₂Cl₂, C1-C4 alcohols such as methanol, C2-C6 ethers, C3-C5 esters such as ethylacetate, and a combination of two or more thereof and a combination of one and water, more preferably from the group consisting of C1-C4 alcohols such as methanol, C3-C6 ketones such as acetone, and a combination of two or more thereof, wherein more preferably, the at least one solvent comprises, more preferably consists of, and C1-C4 alcohol, preferably methanol, or comprises, more preferably consists of, acetone.
64. The solid pharmaceutical composition of any one of embodiments 1 to 63 or 124 to 130, for use in a method for treating hepatitis C in a human.
65. The solid pharmaceutical composition of any one of embodiments 1 to 64 or 124 to 130, for treating hepatitis C in a human.
66. A process for preparing a solid pharmaceutical composition according to any one of embodiments 1 to 65 or 124 to 130, comprising
   (i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
   (ii) processing the mixture obtained from (i) to the solid pharmaceutical composition.
67. The process of embodiment 66, said processing comprising
   (ii) directly processing the mixture obtained from (i) to the pharmaceutical composition.
68. The process of embodiment 66 or 67, wherein after (i) and before (ii), the mixture obtained from (i) is not subjected to any modification.
69. The process of any one of embodiments 66 to 68, wherein (ii) comprise one or more of wet granulation, dry granulation, compression, melting extrusion of the mixture of (i).
70. The process of any one of embodiments 66 to 69, wherein (ii) comprise melting extruding the mixture of (i), preferably hot melting extruding the mixture of (i).
71. The process of any one of embodiments 66 to 70, wherein the mixture obtained from (i) is melt extruded to the pharmaceutical composition according to (ii) at most 168 h, preferably at most 72 h, more preferably at most 24 h after having been obtained from (i), wherein during this period of time, the solid pharmaceutical composition is preferably not subjected to stress conditions of 30° C. and a relative humidity of 75%, more preferably stored under ambient conditions.
72. The process of any one of embodiments 66 to 71, wherein the solid pharmaceutical composition is a solid dispersion.
73. The process of any one of embodiments 66 to 72, wherein in (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.
74. The process of any one of embodiments 66 to 73, wherein (i) and (ii) comprise embedding the sofosbuvir in a matrix comprising, preferably consisting of the at least one pharmaceutically acceptable matrix compound and one or more pharmaceutically acceptable excipient(s), wherein said embedding preferably comprises melting extruding the at least one pharmaceutically acceptable matrix compound and the one or more pharmaceutically acceptable excipient(s) together with the sofosbuvir.
75. The process of embodiment 66 to 73, wherein (i) and (ii) comprise embedding sofosbuvir in a matrix comprising, preferably consisting of the at least one pharmaceutically acceptable matrix compound and at least one pharmaceutical acceptable excipient, by melting extruding the at least one pharmaceutically acceptable matrix compound in solid form and the one or more pharmaceutical acceptable excipient(s) in solid form together with the sofosbuvir in solid form, preferably by a hot-melt extrusion method.
76. The process of any one of embodiments 66 to 75, wherein (ii) comprises melting extruding at a temperature in the range of from 75 to 175° C., preferably in the range of from 90 to 150° C.
77. The process of any one of embodiments 66 to 76, wherein the process after the melting extruding according to (ii), comprises cooling, preferably to a temperature in the range of from 10 to 40° C., preferably in the range of from 20 to 30° C.
78. The process of any one of embodiments 66 to 77, wherein the sofosbuvir in solid form before processing, preferably melting extruding is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
79. The process of any one of embodiments 66 to 78, wherein at least 99 weight-%, preferably at least 99.5 weight-%, more preferably at least 99.9 weight-% of the sofosbuvir comprised in the solid pharmaceutical composition are present in amorphous form directly after preparing the solid composition.
80. A process for preparing a solid pharmaceutical composition according to any one of embodiments 1 to 44 and 124 to 130, comprising
   (i′) preparing a mixture of sofosbuvir and the at least one pharmaceutically acceptable matrix compound and at least one solvent;
   (ii′) subjecting the mixture of (i′) to drying, preferably by lyophilizing the mixture of (i′) or spray-drying the mixture of (i′), obtaining a dried, preferably lyophilize or spray-dried mixture-1;
   (iii′) mixing the mixture-1 of (ii′) with the one or more pharmaceutically acceptable excipient(s), obtaining a mixture-2;
   (iv′) processing the mixture-2 of (iii′) to the solid pharmaceutical composition.
81. The process of embodiment 80, wherein the mixture of (i′) is a solution or a dispersion or a suspension.
82. The process of embodiment 80 or 81, wherein (i′) and (ii′) comprise embedding sofosbuvir in a matrix comprising, preferably consisting of the at least one pharmaceutically acceptable matrix compound, wherein said embedding comprises preparing a mixture of the sofosbuvir, the at least one pharmaceutically acceptable matrix compound and at least one solvent and subjecting said mixture to drying, preferably by lyophilizing the mixture or spray-drying the mixture.
83. The process of embodiments 80 or 81, wherein the at least one solvent is selected from the group consisting of an organic solvent, and a combination of two or more thereof, wherein the organic solvent is preferably selected from the group consisting of a C1-C2 halogenated hydrocarbon, a C1-C4 alcohol, a C3-C6 ketone, a C2-C6 ether, a C3-05 ester, a combination of two or more thereof and a combination of one or more thereof with water.
84. The process of embodiment 83, wherein the at least one solvent is selected from the group consisting of C1-C4 alcohols, C1-C2 halogenated hydrocarbons, C3-C6 ketones, C2-C6 ethers, C3-C5 esters, and a combination of two or more thereof.
85. The process of embodiment 83 or 84, wherein the at least one solvent is selected from the group consisting of acetone, CH₂Cl₂ and methanol, preferably acetone.
86. The process of any one of embodiments 82 to 85, wherein the embedding comprises subjecting the solution to drying, preferably by lyophilizing the solution or spray-drying the solution.
87. The process of any one of embodiments 66 to 87, wherein the at least one matrix compound is selected from the group consisting of hydrophilic water-soluble polymers, silicon-based inorganic adsorbents and a combination of two or more thereof.
87. The process of any one of embodiments 66 to 87, wherein the at least one matrix compound is selected from the group consisting of silicon-based inorganic adsorbents and a combination of two or more thereof and wherein the embedding comprises dispersing the at least one matrix compound in the solution.
88. The process of embodiment 87, wherein the at least one matrix compound has a pH in the range of from 6.0 to 9.0, preferably in the range of from 6.5 to 8.5, more preferably in the range of from 7.0 to 8.0.
89. The process of any one of embodiments 82 to 77, wherein the embedding comprises subjecting the dispersion to drying, preferably filtrating the dispersion or evaporating the dispersion, preferably followed by vacuum drying.
90. The process of any one of embodiments 66 to 89, wherein the solid pharmaceutical composition comprises the sofosbuvir in an amount in the range of from 15 to 95 weight-%, preferably from 30 to 45 weight-%, more preferably from 30 to 40 weight-%, based on the total weight of the solid pharmaceutical composition.
91. The process of any one of embodiments 66 to 90, wherein the at least one matrix compound is selected from the group consisting of hydrophilic polymers, silicon-based inorganic adsorbents and a combination of two or more thereof.
92. The process of any one of embodiments 66 to 91, wherein the silicon-based inorganic adsorbents include, preferably are, one or more of silica and silicates.
93. The process of any one of embodiments 66 to 92, wherein the at least one silicon-based inorganic adsorbent has an oil absorbance in the range of from 1.0 to 5.0 ml/g, preferably in the range of from 1.5 to 4.0 ml/g.
94. The process of any one of embodiments 66 to 93, wherein the at least one silicon-based inorganic adsorbent has a bulk density in the range of from 10 to 600 g/ml, preferably in the range of from 30 to 500 g/ml, more preferably in the range of from 50 to 300 g/ml, more preferably in the range of from 50 to 200 g/ml.
95. The process of any one of embodiments 66 to 94, wherein the at least one silicon-based inorganic adsorbent is selected from the group consisting of silica, silicates, and a combination of two or more thereof, wherein the silica is preferably selected from the group consisting of fumed silica, precipitated silica, gel silica, colloidal silica, and a combination of two or more thereof, and wherein the silicates are preferably aluminosilicates preferably comprising at least one alkali metal element and/or at least one alkaline earth metal element, more preferably at least one alkaline earth metal element, more preferably magnesium, wherein more preferably, at least 90 weight-%, more preferably at least 95 weight-%, more preferably at least 99 weight-% of the at least one silicon-based inorganic adsorbent are present in amorphous form.
96. The process of embodiment 95, wherein the hydrophilic polymers include, preferably are, one or more of polysaccharides, preferably cellulose derivatives such as hydroxyalkylalkylcelullose, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.
97. The process of any one of embodiments 66 to 96 wherein the at least one matrix compound is selected from the group consisting of hydrophilic water-soluble polymers and a combination of two or more thereof.
98. The process of embodiment 97, wherein the at least one hydrophilic water-soluble polymer comprises, preferably consists of, at least one vinyl pyrrolidone-vinyl acetate copolymer.
99. The process of any one of embodiments 97 or 98, wherein the at least one hydrophilic water-soluble polymer comprises, preferably consists of, copovidone.
100. The process of any one of embodiments 97 to 99, wherein the weight average molecular weight (M_{w}) of the at least one hydrophilic water-soluble polymer is in the range of from 20 to 100 kDa, preferably in the range of from 30 to 85 kDa, more preferably in the range of from 40 to 75 kDa.
101. The process of embodiment 97, wherein the at least one hydrophilic water-soluble polymer comprises, preferably consists of a cellulose derivative selected from the group consisting of hydroxyalkylalkylcelluloses and a mixture of two or more thereof, the at least one hydrophilic water-soluble polymer preferably comprising, more preferably consisting of, hydroxypropylmethylcellulose (HPMC).
102. The process of embodiment 101, wherein the cellulose derivative has a degree of substitution (DS) in the range of from 0.3 to 2.8, preferably in the range of from 0.6 to 2.5, more preferably in the range of from 1.0 to 2.3, more preferably in the range of from 1.3 to 2.0.
103 The process of embodiment 101 or 102, wherein the weight average molecular weight (M_{w}) of the cellulose derivative is in the range of from 7 to 225 kDa, preferably in the range of from 7 to 100 kDa, more preferably in the range of from 7 to 30 kDa. 104. The process of any one of embodiments 66 to 104, wherein at least 99.5 weight-%, preferably at least 99.9 weight-% of the sofosbuvir comprised in the composition are present in amorphous form.
105. The process of any one of embodiments 66 to 104 wherein at least 99.5 weight-%, preferably at least 99.9 weight-% of the solid pharmaceutical composition consists of the sofosbuvir the at least one matrix compound and one or more pharmaceutically acceptable excipient(s).
106. The process of any one of embodiments 66 to 105 wherein from 30 to 60 weight-% or from 35 to 55 weight-% or from 40 to 50 weight-% of the solid pharmaceutical composition consists of sofosbuvir based on the total weight of the solid pharmaceutical composition.
107. The process of any one of embodiments 66 to 106 wherein from 30 to 45 weight-%, preferably from 30 to 40 weight-%, more preferably 30 weight-% of the solid pharmaceutical composition consists of sofosbuvir based on the total weight of the solid pharmaceutical composition.
108. The process of any one of embodiments 66 to 107, wherein from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.
109. The process of any one of embodiments 66 to 108, wherein the solid pharmaceutical composition comprises less than 0.1 weight-%, preferably less than 0.01 weight-%, more preferably less than 0.001 weight-% of a surfactant.
110. The process of any one of embodiments 66 to 109 wherein the solid pharmaceutical composition is a solid dispersion.
111. The process of any one of embodiments 66 to 110, wherein the solid pharmaceutical composition is an oral dosage form, preferably a tablet.
112. The process of any one of embodiments 66 to 111, wherein the at least one matrix compound is selected from the group consisting of hydrophilic water-soluble polymers, silicon-based inorganic adsorbents and a combination of two or more thereof.
113. The process of any one of embodiment 66 to 112, wherein the one or more pharmaceutically acceptable excipient(s) are different from the at least one pharmaceutically acceptable matrix compound comprised in the solid pharmaceutical composition, preferably wherein the one or more pharmaceutically acceptable excipient(s) are not a pharmaceutically acceptable matrix compound as defined in any of embodiment 3 to 8.
114. The process of any of embodiments 66 to 113, wherein the one or more pharmaceutically acceptable excipient(s) includes one or more of at least one of a diluent, at least one disintegrant, at least one glidant, optionally at least one lubricant, and a combination of two or more thereof.
115. The process of embodiment 66 to 114, wherein the one or more pharmaceutically acceptable excipient(s) preferably comprise, more preferably are, a combination of at least one a diluent, at least one disintegrant, at least one glidant, and at least one lubricant.
116. The process of embodiment 115, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate,
117. The process of any one of embodiments 66 to 116, wherein the one or more pharmaceutically acceptable excipient(s) comprise, preferably are, a combination of mannitol, microcrystalline cellulose, croscarmello se sodium, colloidal silica, optionally magnesium stearate.
118. The process of any one of embodiments 66 to 117, for preparing the solid pharmaceutical formulation of any one of embodiments 1 to 65 or 124 to 130.
119. The process of any one of embodiments 66 to 118, wherein the solid pharmaceutical composition is a tablet.
120. A solid pharmaceutical composition obtained or obtainable by a process according to any one of embodiments 66 to 119 or 131 to 137 or 140 or 141.
121. A tablet obtained or obtainable by a process according to any one of embodiments 66 to 120.
122. Use of a solid pharmaceutical composition according to any one of embodiments 1 to 65 and 124 to 130 for the preparation of a medicament for treating hepatitis C in a human.
123. A method for treating hepatitis C comprising administering a solid pharmaceutical composition according to any one of embodiments 1 to 65 and 124 to 130 to a human in need thereof.
124. The solid pharmaceutical composition of any of embodiments 1 to 65, 120, wherein at least 1.5 weight-%, preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
125. The solid pharmaceutical composition of any one of embodiments 1 to 65, 120, 123, 124, wherein at least 15 weight-%, preferably from 20 to 30 weight-%, more preferably from 22 to 26 weight-% of the solid composition consists of the diluent, based on the total weight of the solid pharmaceutical composition.
126. The solid pharmaceutical composition of any one of embodiments 1 to 65, 120, 123 to 125, wherein at least 16.5 weight-%, preferably from 22 to 36 weight-%, more preferably from 27.5 to 32.5 weight-% of the composition consists of a disintegrant, based on the total weight of the solid pharmaceutical composition.
127. The solid pharmaceutical composition of any one of embodiments 1 to 65, 120, 123 to 126, wherein at least 15 weight-%, preferably from 20 to 30 weight-%, more preferably from 24 to 28 weight-% of the composition consists of the first disintegrant, based on the total weight of the solid pharmaceutical composition.
128. The solid pharmaceutical composition of any one of embodiments 1 to 65, 120, 123 to 127, wherein at least 1.5 weight-%, preferably from 2 to 6 weight-%, more preferably from 2.5 to 5.5 weight-% of the composition consists of the second disintegrant, based on the total weight of the solid pharmaceutical composition.
129. The solid pharmaceutical composition of any one of embodiments 1 to 65, 120, 123 to 128, wherein at least 0.2 weight-%, preferably from 0.5 to 7 weight-%, more preferably from 0.8 to 5 weight-% of the composition consists of the glidant, based on the total weight of the solid pharmaceutical composition.
130. The solid pharmaceutical composition of any one of embodiments 1 to 65, 120, 123 to 129, wherein at least 0.5 weight-%, preferably from 1 to 6 weight-%, more preferably from 1.4 to 2 weight-% of the composition consists of the lubricant, based on the total weight of the solid pharmaceutical composition.
131. The process of any of embodiments 66 to 119, wherein at least 1.5 weight-%, preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
132. The process of any one of embodiments 66 to 119 and 131, wherein at least 15 weight-%, preferably from 20 to 30 weight-%, more preferably from 22 to 26 weight-% of the solid composition consists of the diluent, based on the total weight of the solid pharmaceutical composition.
133. The process of any one of embodiments 66 to 119, 131 and 132, wherein at least 16.5 weight-%, preferably from 22 to 36 weight-%, more preferably from 27.5 to 32.5 weight-% of the composition consists of a disintegrant, based on the total weight of the solid pharmaceutical composition.
134. The process of any one of embodiments 66 to 119 and 131 to 133, wherein at least 15 weight-%, preferably from 20 to 30 weight-%, more preferably from 24 to 28 weight-% of the composition consists of the first disintegrant, based on the total weight of the solid pharmaceutical composition.
135. The process of any one of embodiments 66 to 119 and 131 to 134, wherein at least 1.5 weight-%, preferably from 2 to 6 weight-%, more preferably from 2.5 to 5.5 weight-% of the composition consists of the second disintegrant, based on the total weight of the solid pharmaceutical composition.
136. The process of any one of embodiments 66 to 119 and 131 to 135, wherein at least 0.2 weight-%, preferably from 0.5 to 7 weight-%, more preferably from 0.8 to 5 weight-% of the composition consists of the glidant, based on the total weight of the solid pharmaceutical composition.
137. The process of any one of embodiments 66 to 119 and 131 to 136, wherein at least 0.5 weight-%, preferably from 1 to 6 weight-%, more preferably from 1.4 to 2 weight-% of the composition consists of the lubricant, based on the total weight of the solid pharmaceutical composition.
138. The solid pharmaceutical composition of any one of embodiments 1 to 65, 120, 123 to 130, wherein the pharmaceutically acceptable matrix compound is optional.
139. The solid pharmaceutical composition of any one of embodiments 1 to 65, 120, 123 to 130 or 138 wherein the solid pharmaceutical composition does not comprise the at least one pharmaceutically acceptable matrix compound.
140. The process of any one of embodiments 66 to 119 and 131 to 137, wherein the pharmaceutically acceptable matrix compound is optional.
141. The process of any one of embodiments 66 to 119 and 131 to 137 or 140, wherein the solid pharmaceutical composition does not comprise the at least one pharmaceutically acceptable matrix compound.

According to a further aspect, the present invention is illustrated by the following embodiments and combinations of embodiments resulting from the given dependencies and back-references:
1′. A solid pharmaceutical composition wherein the solid pharmaceutical composition comprises sofosbuvir according to formula (I)

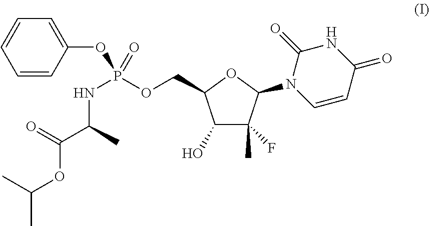 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s) and wherein the at least one pharmaceutically acceptable matrix compound comprises, preferably consists of, at least one vinyl pyrrolidone-vinyl acetate copolymer.
2′. The solid pharmaceutical composition of embodiment 1′, wherein the at least one pharmaceutically acceptable matrix compound comprises, preferably consists of copovidone.
3′. The solid pharmaceutical composition of embodiment 1, wherein the solid pharmaceutical composition comprises the sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.
4′. The solid pharmaceutical composition of any one of embodiments 1′ to 3′, wherein at least 99.5 weight-%, preferably at least 99.9 weight-% of the sofosbuvir comprised in the composition are present in amorphous form.
5′. The solid pharmaceutical composition of any one of embodiments 1′ to 4′, wherein at least 99.5 weight-%, preferably at least 99.9 weight-% of the solid pharmaceutical composition consists of the sofosbuvir the at least one matrix compound and one or more pharmaceutically acceptable excipient(s).
6′. The solid pharmaceutical composition of any one of embodiments 1′ to 5′, wherein the solid pharmaceutical composition comprises less than 0.1 weight-%, preferably less than 0.01 weight-%, more preferably less than 0.001 weight-% of a surfactant.
7′. The solid pharmaceutical composition of any one of embodiments 1′ to 6′ wherein the solid pharmaceutical composition is a solid dispersion.
8′. The solid pharmaceutical composition of any one of embodiments 1′ to 7′, being an oral dosage form, preferably a tablet.
9′. The solid pharmaceutical composition of any one of embodiments 1′ to 8′, further comprising, in addition to the sofosbuvir, one or more further HCV agents including one or more of ledipasvir according to formula (II)

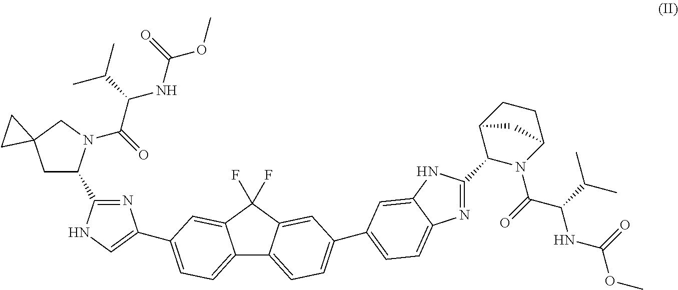 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

and daclatasvir of formula (III)

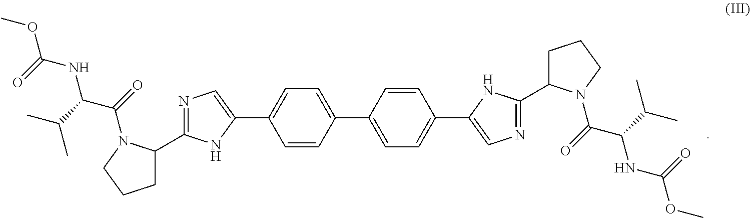

10′. The solid pharmaceutical composition of any one of embodiments 1′ to 9′, wherein the solid pharmaceutical composition consists of sofosbuvir, the at least one pharmaceutically acceptable matrix compound, the one or more pharmaceutically acceptable excipient(s) and optionally the one or more further HCV agents.
11′. The solid pharmaceutical composition of any one of embodiments 1′ to 10′, wherein the one or more pharmaceutically acceptable excipient(s) comprise one or more of at least one of a diluent, at least one disintegrant, at least one glidant, optionally at least one lubricant, and combinations of two or more thereof.
12′. The solid pharmaceutical composition of embodiment 11′, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
13′. The solid pharmaceutical composition of embodiment 12′, wherein the at least one diluent is mannitol.
14′. The solid pharmaceutical composition of any of embodiments 11′ to 13′, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
15′. The solid pharmaceutical composition of embodiment 14′, wherein the at least one disintegrant is selected from croscarmellose sodium, such as Ac-Di-Sol® croscarmellose sodium.
16′. The solid pharmaceutical composition of any one of embodiments 11′ to 15′, wherein the at least one glidant includes one or more of colloidal silicon dioxide, talc, starch, starch derivatives.
17′. The solid pharmaceutical composition of embodiment 16′, wherein the at least one disintegrant is selected from croscarmellose sodium, such as Ac-Di-Sol® croscarmellose sodium.
18′. The solid pharmaceutical composition of any one of embodiments 11′ to 17′, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
19′. The solid pharmaceutical composition of embodiment 18′, wherein the at least one glidant is colloidal silicon dioxide.
20′. The solid pharmaceutical composition of any one of embodiments 1′ to 19′, wherein the one or more pharmaceutically acceptable excipient(s) comprise, preferably are, a combination of at least one a diluent, at least one disintegrant, at least one glidant, and optionally at least one lubricant.
21′. The solid pharmaceutical composition of embodiment 20′, comprising
   from 20 to 30 weight-%, preferably from 22 to 26 weight-% diluent,
   from 20 to 30 weight-%, preferably from 24 to 28 weight-% first disintegrant,
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
22′. The solid pharmaceutical composition of embodiment 20′ or 21′, comprising
   from 30 to 45 weight-%, preferably from 30 to 40 weight-% sofosbuvir,
   from 5 to 15 weight-%, preferably from 7 to 13 weight-% of the pharmaceutically
   acceptable matrix compound,
   from 20 to 30 weight-%, preferably from 22 to 26 weight-% diluent,
   from 20 to 30 weight-%, preferably from 24 to 28 weight-% first disintegrant
   from 2 to 6 weight-%, preferably from 2.5 to 5.5 weight-% second disintegrant,
   from 0.5 to 7 weight-%, preferably from 0.8 to 5 weight-% glidant,
   optionally from 1 to 6 weight-%, preferably from 1.4 to 2 weight-% lubricant,
   in each based on the total weight of the solid pharmaceutical composition.
23′. The solid pharmaceutical composition of any one of embodiments 20′ to 22′, wherein the one or more pharmaceutically acceptable excipient(s) comprise, preferably are, a combination of mannitol, microcrystalline cellulose, croscarmellose sodium, silica dioxide, magnesium stearate.
24′. The solid pharmaceutical composition of embodiment 23′, comprising
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium
   stearate,
   in each based on the total weight of the solid pharmaceutical composition.
25′. The solid pharmaceutical composition of embodiment 24′, comprising
   from 30 to 45 weight-%, preferably from 30 to 40 weight-% sofosbuvir,
   from 5 to 15 weight-%, preferably from 7 to 13 weight-% copovidone,
   from 20 to 30 weight-%, preferably from 22 to 26 weight-% mannitol,
   from 20 to 30 weight-%, preferably from 24 to 28 weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   optionally from 1 to 6 weight-%, preferably from 1.4 to 2 weight-% magnesium stearate,
   in each based on the total weight of the solid pharmaceutical composition.
26′. The solid pharmaceutical composition of embodiment 27′, comprising
   from 30 to 40 weight-% sofosbuvir,
   from 7 to 13 weight-% copovidone,
   from 22 to 26 weight-% mannitol,
   from 24 to 28 weight-% microcrystalline cellulose,
   from 2.5 to 5.5 weight-% croscarmellose sodium,
   from 0.8 to 5 weight-% colloidal silica,
   optionally from 1.4 to 2 weight-% magnesium stearate,
   in each based on the total weight of the solid pharmaceutical composition, wherein the individual contents add up to 100%. 27′. The solid pharmaceutical composition of any one of embodiments 1′ to 26′ which is a tablet or a coated tablet.
28′. The solid pharmaceutical composition of embodiment 27′, wherein the tablet has a weight in the range of from 900 mg to 1300 mg
29′. The solid pharmaceutical composition of any one of embodiments 1′ to 28′, obtainable or obtained by a process comprising
   (i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
   (ii) processing the mixture obtained from (i) to the solid pharmaceutical composition, wherein preferably the solid pharmaceutical composition is a tablet.
30′ The solid pharmaceutical composition of embodiment 29′, said process comprising
   (ii) directly processing the mixture obtained from (i) to the pharmaceutical composition.
31′ The solid pharmaceutical composition of embodiment 29′ or 30′, wherein (ii) comprise one or more of wet granulation, dry granulation, compression, melting extrusion of the mixture of (i).
32′. The solid pharmaceutical composition of any one of embodiments 29′ to 31′, wherein (ii) comprise melting extruding the mixture of (i), preferably hot melting extruding the mixture of (i).
33′. The solid pharmaceutical composition of any one of embodiments 1′ to 32′, wherein the sofosbuvir before melt extrusion is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form, preferably crystalline form is form 1.
34′. The solid pharmaceutical composition of any one of embodiments 1 to 33′, wherein at least 99 weight-%, preferably at least 99.5 weight-%, more preferably at least 99.9 weight-% of the sofosbuvir comprised in the solid pharmaceutical composition are present in amorphous form.
35′. The solid pharmaceutical composition of any one of embodiments 1′ to 34′, wherein the melt extrusion is carried out at a temperature of at least 100° C., preferably at least 150° C.
36′. The solid pharmaceutical composition of any one of embodiments 1′ to 35′ or 67′ to 73′, for use in a method for treating hepatitis C in a human.
37′. The solid pharmaceutical composition of any one of embodiments 1′ to 36′ or 67′ to 73′ for treating hepatitis C in a human.
38′. A process for preparing a solid pharmaceutical composition according to any one of embodiments 1′ to 37′ or 67′ to 73′, comprising
   (i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
   (ii) processing the mixture obtained from (i) to the solid pharmaceutical composition.
39′. The process of embodiment 38′, said processing comprising
   (ii) directly processing the mixture obtained from (i) to the pharmaceutical composition.
40′. The process of embodiment 38′ or 39′, wherein (ii) comprise one or more of wet granulation, dry granulation, compression, melting extrusion of the mixture of (i).
41′. The process of any one of embodiments 38′ to 40′, wherein (ii) comprise melting extruding the mixture of (i), preferably hot melting extruding the mixture of (i).
42′. The process of any one of embodiments 38′ to 41′, wherein the solid pharmaceutical composition is a solid dispersion.
43′. The process of any one of embodiments 1′, wherein in (i) the weight ratio of the sofosbuvir relative to the at least one matrix compound is at least 10:1, preferably in the range of from 6:1 to 1:1 more preferably in the range of from 5:1 to 2:1, more preferably in the range of from 4.5:1 to 2.6:1.
44′. The process of any one of embodiments 40′ to 43′, wherein (ii) comprises melting extruding at a temperature in the range of from 75 to 175° C., preferably in the range of from 90 to 150° C.
45′. The process of any one of embodiments 40′ to 44′ wherein the process after the melting extruding according to (ii), comprises cooling, preferably to a temperature in the range of from 10 to 40° C., preferably in the range of from 20 to 30° C.
46′. The process of any one of embodiments 38′ to 45′, wherein the sofosbuvir before processing, preferably melting extruding is sofosbuvir in at least one crystalline form or in amorphous form or as a mixture of at least one crystalline form and amorphous form, wherein preferably at least 95 weight-%, preferably at least 99 weight-%, more preferably at least 99.9 weight-% of the sofosbuvir are present in at least one crystalline form.
47′ The process of any one of embodiments 38′ to 46′, wherein at least 99 weight-%, preferably at least 99.5 weight-%, more preferably at least 99.9 weight-% of the sofosbuvir comprised in the solid pharmaceutical composition are present in amorphous form directly after preparing the solid composition.
48′. The process of any one of embodiments 38′ to 47′ wherein the at least one pharmaceutically acceptable matrix compound comprises, preferably consists of, at least one vinyl pyrrolidone-vinyl acetate copolymer.
49′. The process of any one of embodiments 38′ to 48′, wherein the at least one hydrophilic water-soluble polymer comprises, preferably consists of, copovidone.
50′. The process of any one of embodiments 38′ to 49′ wherein at least 99.5 weight-%, preferably at least 99.9 weight-% of the sofosbuvir comprised in the composition are present in amorphous form.
51′. The process of any one of embodiments 38′ to 50′ wherein at least 99.5 weight-%, preferably at least 99.9 weight-% of the solid pharmaceutical composition consists of the sofosbuvir the at least one matrix compound and one or more pharmaceutically acceptable excipient(s).
52′. The process of any one of embodiments 38′ to 51′, wherein from 15 to 95 weight-%, preferably from 30 to 45 weight-%, more preferably from 30 to 40 weight-% of the solid pharmaceutical composition consists of sofosbuvir based on the total weight of the solid pharmaceutical composition.
53′. The process of any one of embodiments 38′ to 52′, wherein from 3 to 15 weight-%, more preferably from 3 to 13 weight-%, more preferably from 3 to 5 weight-% of the solid pharmaceutical composition consists of the at least one pharmaceutically acceptable matrix compound based on the total weight of the solid pharmaceutical composition.
54′. The process of any one of embodiments 38′ to 53′, wherein the solid pharmaceutical composition comprises less than 0.1 weight-%, preferably less than 0.01 weight-%, more preferably less than 0.001 weight-% of a surfactant.
55′. The process of any one of embodiments 38′ to 54′, wherein the solid pharmaceutical composition is a solid dispersion.
56′. The process of any one of embodiments 38′ to 55′, wherein the solid pharmaceutical composition is an oral dosage form, preferably a tablet.
57′. The process of embodiment 38′ to 56′ wherein the one or more pharmaceutically acceptable excipient(s) includes one or more of at least one of a diluent, at least one disintegrant, at least one glidant, optionally at least one lubricant, and a combination of two or more thereof.
58′. The process of embodiment 38′ to 57′ wherein the one or more pharmaceutically acceptable excipient(s) preferably comprise, more preferably are, a combination of at least one a diluent, at least one disintegrant, at least one glidant, and at least one lubricant.
59′. The process of embodiment 58′, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
59′. The process of any one of embodiments 38′ to 58′ wherein the one or more pharmaceutically acceptable excipient(s) comprise, preferably are, a combination of mannitol, microcrystalline cellulose, croscarmellose sodium, colloidal silica, optionally magnesium stearate.
60′. The process of any one of embodiments 38′ to 59′ for preparing the solid pharmaceutical formulation of any one of embodiments 1 to 37′.
61′. The process of any one of embodiments 38′ to 60′, wherein the solid pharmaceutical composition is a tablet.
62′. A solid pharmaceutical composition obtained or obtainable by a process according to any one of embodiments 38′ to 61′ and 74′ to 80′.
63′. A tablet obtained or obtainable by a process according to any one of embodiments 38′ to 61′ or 74′ to 80′.
64′. Use of a solid pharmaceutical composition according to any one of embodiments 1′ to 37′ or 62′ of 67′ to 73′ for the preparation of a medicament for treating hepatitis C in a human.
65′. A method for treating hepatitis C comprising administering a solid pharmaceutical composition according to any one of embodiments 1′ to 37′ or 62′ or 67′ to 73′ to a human in need thereof.
66′. The solid pharmaceutical composition of any one of embodiments 1′ to 37′ and 67′ to 73′ obtained or obtainable by melt extrusion, preferably by hot-melt extrusion.
67′. The solid pharmaceutical composition of any of embodiments 1′ to 37′, wherein at least 1.5 weight-% preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
68′. The solid pharmaceutical composition of any one of embodiments 1′ to 37′ and 67′, wherein at least 15 weight-%, preferably from 20 to 30 weight-%, more preferably from 22 to 26 weight-% of the solid composition consists of the diluent, based on the total weight of the solid pharmaceutical composition.
69′. The solid pharmaceutical composition of any one of embodiments 1′ to 37′ and 67′ to 68′, wherein at least 16.5 weight-%, preferably from 22 to 36 weight-%, more preferably from 27.5 to 32.5 weight-% of the composition consists of a disintegrant, based on the total weight of the solid pharmaceutical composition.
70′. The solid pharmaceutical composition of any one of embodiments 1′ to 37′ and 67′ to 69′, wherein at least 15 weight-%, preferably from 20 to 30 weight-%, more preferably from 24 to 28 weight-% of the composition consists of the first disintegrant, based on the total weight of the solid pharmaceutical composition.
71′. The solid pharmaceutical composition of any one of embodiments 1′ to 37′ and 67′ to 70′, wherein at least 1.5 weight-%, preferably from 2 to 6 weight-%, more preferably from 2.5 to 5.5 weight-% of the composition consists of the second disintegrant, based on the total weight of the solid pharmaceutical composition.
72′. The solid pharmaceutical composition of any one of embodiments 1′ to 37′ and 67′ to 71′, wherein at least 0.2 weight-%, preferably from 0.5 to 7 weight-%, more preferably from 0.8 to 5 weight-% of the composition consists of the glidant, based on the total weight of the solid pharmaceutical composition.
73′. The solid pharmaceutical composition of any one of embodiments 1′ to 37′ and 67′ to 72′, wherein at least 0.5 weight-%, preferably from 1 to 6 weight-%, more preferably from 1.4 to 2 weight-% of the composition consists of the lubricant, based on the total weight of the solid pharmaceutical composition.
74′. The process of any of embodiments 38′ to 61′, wherein at least 1.5 weight-% preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
75′. The process of any one of embodiments 38′ to 61′ and 74′, wherein at least 15 weight-%, preferably from 20 to 30 weight-%, more preferably from 22 to 26 weight-% of the solid composition consists of the diluent, based on the total weight of the solid pharmaceutical composition.
76′. The process of any one of embodiments 38′ to 61′ and 74′ and 75′, wherein at least 16.5 weight-%, preferably from 22 to 36 weight-%, more preferably from 27.5 to 32.5 weight-% of the composition consists of a disintegrant, based on the total weight of the solid pharmaceutical composition.
77′. The process of any one of embodiments 38′ to 61′ and 74′ to 76′, wherein at least 15 weight-%, preferably from 20 to 30 weight-%, more preferably from 24 to 28 weight-% of the composition consists of the first disintegrant, based on the total weight of the solid pharmaceutical composition.
78′. The process of any one of embodiments 38′ to 61′ and 74′ and 77′ wherein at least 1.5 weight-%, preferably from 2 to 6 weight-%, more preferably from 2.5 to 5.5 weight-% of the composition consists of the second disintegrant, based on the total weight of the solid pharmaceutical composition.
79′. The process of any one of embodiments 38′ to 61′ and 74′ and 78′, wherein at least 0.2 weight-%, from 0.5 to 7 weight-%, more preferably from 0.8 to 5 weight-% of the composition consists of the glidant, based on the total weight of the solid pharmaceutical composition.
80′. The process of any one of embodiments 38′ to 61′ and 74′ and 79′, wherein at least 0.5 weight-%, preferably from 1 to 6 weight-%, more preferably from 1.4 to 2 weight-% of the composition consists of the lubricant, based on the total weight of the solid pharmaceutical composition.

According to a further aspect, the present invention is illustrated by the following embodiments and combinations of embodiments resulting from the given dependencies and back-references:
1*. A solid pharmaceutical composition wherein the solid pharmaceutical composition comprises sofosbuvir according to formula (I)

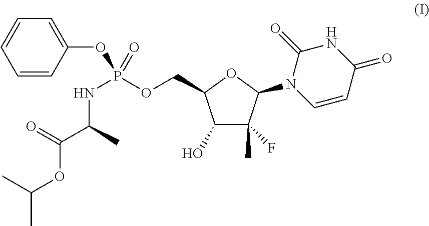 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]

and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir and the one or more pharmaceutically acceptable excipient(s).
2*. The solid pharmaceutical composition of embodiment 1* wherein at least 99.5 weight-%, preferably at least 99.9 weight-% of the sofosbuvir comprised in the composition are present in amorphous form.
3*. The solid pharmaceutical composition of embodiment 1* or 2*, wherein the solid pharmaceutical composition comprises sofosbuvir in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.
4*. The solid pharmaceutical composition of any one of embodiments 1* to 3*, wherein from 20 to 45 weight-%, more preferably from 20 to 40 weight-%, more preferably from 30 to 40 weight-%, more preferably 30 weight-%, based on the total weight of the solid pharmaceutical composition.
5*. The solid pharmaceutical composition of any one of embodiments 1* to 4*, wherein 30 or 35 or 40 or 45 weight-% of the solid pharmaceutical composition consists of sofosbuvir based on the total weight of the solid pharmaceutical composition.
6*. The solid pharmaceutical composition of any one of embodiments 1* to 5*, wherein at least 99.5 weight-%, preferably at least 99.9 weight-% of the solid pharmaceutical composition consists of the sofosbuvir and one or more pharmaceutically acceptable excipient(s).
7*. The solid pharmaceutical composition of any one of embodiments 1* to 6*, being an oral dosage selected from the group consisting of a granule, a capsule such as a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet and an extrudate, preferably the solid pharmaceutical composition is a tablet or a coated tablet.
8*. The solid pharmaceutical composition of any one of embodiments 1* to 7*, further comprising, in addition to the sofosbuvir, one or more further HCV agents including one or more of ledipasvir according to formula (II)

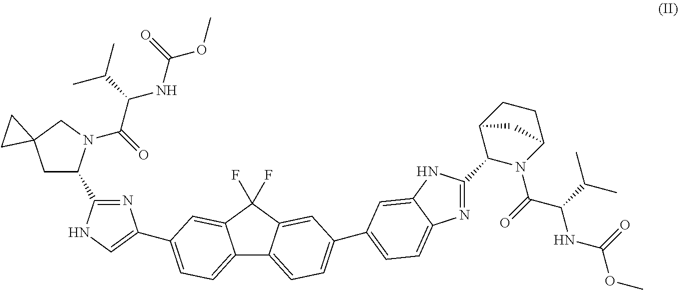 [SMILES: COC(=O)N[C@H](C(=O)N1CC2(CC2)C[C@H]1c1nc(-c2ccc3c(c2)C(F)(F)c2cc(-c4ccc5nc([C@@H]6[C@H]7CC[C@H](C7)N6C(=O)[C@@H](NC(=O)OC)C(C)C)[nH]c5c4)ccc2-3)c[nH]1)C(C)C]

and daclatasvir of formula (III)

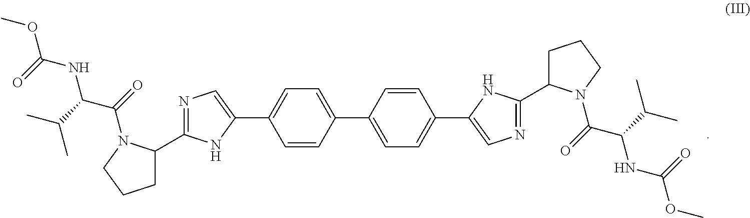

9*. The solid pharmaceutical composition of any one of embodiments 1* to 8*, wherein the solid pharmaceutical composition consists of sofosbuvir, and the one or more pharmaceutically acceptable excipient(s) and optionally the one or more further HCV agents.
10*. The solid pharmaceutical composition of any one of embodiments 1* to 9*, wherein the one or more pharmaceutically acceptable excipient(s) comprise one or more of at least one diluent, of at least one disintegrant, of at least one glidant, of at least one lubricant and optionally of at least one coating agent, and combinations of two or more thereof.
11*. The solid pharmaceutical composition of embodiment 10*, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate.
12*. The solid pharmaceutical composition of embodiment 11*, wherein the at least one diluent is selected from the group consisting of mannitol and microcrystalline cellulose or mixture thereof, wherein mannitol is preferably dry mannitol.
13*. The solid pharmaceutical composition of any one of embodiments 10* to 12*, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates.
14*. The solid pharmaceutical composition of embodiment 13*, wherein the at least one disintegrant is selected from the group consisting of colloidal silicon dioxide, croscarmellose sodium, microcrystalline cellulose, preferably is croscarmellose sodium such as Ac-Di-Sol.
15*. The solid pharmaceutical composition of any one of embodiments 10* to 14*, wherein the at least one glidant includes one or more of colloidal silicon dioxide, talc, starch, starch derivatives.
16*. The solid pharmaceutical composition of embodiment 15*, wherein the at least one glidant is silicon dioxide.
17*. The solid pharmaceutical composition of any one of embodiments 10* to 16*, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate.
18*. The solid pharmaceutical composition of embodiment 17*, wherein the at least one glidant is magnesium stearate.
19*. The solid pharmaceutical composition of any one of embodiments 10* to 18*, wherein the at least one coating agent includes one or more of from the group consisting of aqueous film coating agent.
20*. The solid pharmaceutical composition of embodiment 19*, wherein the at least one aqueous film coating agent is selected from the group consisting of OPADRY and OPADRY II.
21*. The solid pharmaceutical composition of any one of embodiments 1* to 20*, wherein the one or more pharmaceutically acceptable excipient(s) comprise, preferably are, a combination of at least one a diluent, at least one disintegrant, at least one glidant, at least one lubricant and optionally at least one coating agent.
22*. The solid pharmaceutical composition of any one of embodiments 10* to 21*, comprising
   from 25 to 35 weight-%, preferably from 25 to 35 weight-% diluent,
   from 25 to 40 weight-%, preferably from 25 to 35 weight-% disintegrant,
   from 0.5 to 7 weight-%, preferably from 0.8 to 5 weight-% glidant,
   from 1 to 6 weight-%, preferably from 1.4 to 2 weight-% lubricant,
   in each based on the total weight of the solid pharmaceutical composition.
23*. The solid pharmaceutical composition of any one of embodiments 10* to 22*, comprising
   from 30 to 45 weight-%, preferably from 30 to 40 weight-% sofosbuvir,
   from 25 to 35 weight-%, preferably from 25 to 30 weight-% diluent,
   from 25 to 40 weight-%, preferably from 25 to 30 weight-% disintegrant,
   from 0.5 to 7 weight-%, preferably from 0.8 to 5 weight-% glidant,
   from 1 to 6 weight-%, preferably from 1.4 to 2 weight-% lubricant, and optionally
   from 1 to 3.5 preferably from 2 to 3 weight-% aqueous film coating agent,
   in each based on the total weight of the solid pharmaceutical composition.
24*. The solid pharmaceutical composition of any one of embodiments 1 to 23*, wherein the one or more pharmaceutically acceptable excipient(s) comprise, preferably are, a combination of mannitol, microcrystalline cellulose, croscarmellose sodium, silica dioxide, magnesium stearate and optionally a aqueous film coating agent such as Opadry II.
25*. The solid pharmaceutical composition of any of embodiments 1* to 24*, comprising
   from 25 to 35 weight-%, preferably from 25 to 30 weight-% mannitol,
   from 25 to 34 weight-%, preferably from 25 to 30-% weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   from 1 to 6 weight-%, preferably from 1.4 to 2 weight-% magnesium stearate, and
   optionally from 1.5 to 3.5 weight-% a coating agent, preferably an aqueous film coating agent,
   in each based on the total weight of the solid pharmaceutical composition.
26*. The solid pharmaceutical composition of any one of embodiments 1* to 25*, comprising
   from 25 to 60 weight-%, preferably from 30 to 45 weight-% sofosbuvir,
   from 25 to 35 weight-%, preferably from 25 to 30 weight-% mannitol,
   from 25 to 36 weight-%, preferably from 25 to 30-% weight-% microcrystalline cellulose,
   from 2 to 6 weight-%, preferably from 2.5 to 5.5 weight-% croscarmellose sodium,
   from 0.5 to 7 weight-%, preferably from 0.8 to 5 weight-% silica dioxide,
   from 1 to 6 weight-%, preferably from 1.4 to 2 weight-% magnesium stearate, and
   optionally from 1.5 to 3.5 weight-% coating agent, preferably an aqueous film coating agent, preferably Opadry II
   in each based on the total weight of the solid pharmaceutical composition.
27*. The solid pharmaceutical composition of embodiment 25* or 26*, comprising
   from 30 to 40 weight-% sofosbuvir,
   from 26 to 29 weight-% mannitol,
   from 26 to 28 weight-% microcrystalline cellulose,
   from 3.5 to 5.5 weight-% croscarmellose sodium,
   from 0.8 to 4 weight-% colloidal silica,
   optionally from 1.4 to 2 weight-% magnesium stearate,
   optionally from 2.5 to 3.5 weight-% coating agent, preferably an aqueous film coating agent,
   in each based on the total weight of the solid pharmaceutical composition, wherein the individual contents add up to 100%.
28*. The solid pharmaceutical composition of any one of embodiments 1* to 27* which is a tablet preferably selected from the group consisting of an uncoated tablet, a coated tablet, an effervescent tablet, a soluble tablet, a dispersible tablet, an orodispersible tablet, a tablet for use in the mouth, a chewable tablet preferably the solid pharmaceutical composition is a tablet or a coated tablet
29*. The solid pharmaceutical composition of embodiments 28*, wherein the tablet is a coated tablet.
30*. The solid pharmaceutical composition of embodiments 28* or 30* wherein the oral dosage form, preferably the tablet or the coated tablet has a weight in the range of from 900 mg to 1300 mg.
31*. The solid pharmaceutical composition of any one of embodiments 1* to 30*, obtainable or obtained by a process comprising
   (i) providing amorphous sofosbuvir.
   (ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
   (iii) processing the mixture obtained from (ii) to the solid pharmaceutical composition.
32*. The solid pharmaceutical composition of any one of embodiments 1* to 30*, obtainable or obtained by a process comprising
   (i) providing amorphous sofosbuvir by rapid drying, more preferably by spray drying a solution comprising sofosbuvir and one or more solvents;
   (ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
   (iii) processing the mixture obtained from (ii) to the solid pharmaceutical composition.
33*. The solid pharmaceutical composition of embodiment 31* or 32* wherein (iii) preferably consists of
   (iii) directly processing the mixture obtained from (ii) to the pharmaceutical composition.
34*. The solid pharmaceutical composition of any one of embodiments 31* to 33*, wherein the mixture obtained from (ii) is processed to the pharmaceutical composition according to (iii) at most 168 h, preferably at most 72 h, more preferably at most 24 h after having been obtained from (ii), wherein during this period of time, the mixture is preferably not subjected to stress conditions of 30° C. and a relative humidity of 75%, more preferably stored under ambient conditions.
35*. The solid pharmaceutical composition of any one of embodiments 31* to 34*, wherein (iii) comprise one or more of wet granulation, dry granulation, compression, melting extrusion of the mixture of (ii).
36*. The solid pharmaceutical composition of any one of embodiments 31* to 35*, wherein (iii) comprise dry granulation.
37*. The solid pharmaceutical composition of any one of embodiments 31* to 36*, wherein (iii) further comprises coating the solid pharmaceutical composition.
38*. The solid pharmaceutical composition of any one of embodiments 31* to 37*, wherein (i) comprises
   (i′) preparing a solution of sofosbuvir and one or more solvents.
39*. The solid pharmaceutical composition of any one of embodiments 31* to 39*, wherein the one or more solvents is selected from the group consisting of an organic solvent, and a combination of two or more thereof.
40*. The solid pharmaceutical composition of embodiment 39*, wherein the organic solvent is selected from the group consisting of a C1-C2 halogenated hydrocarbon such as CH₂Cl₂, a C1-C4 alcohol, such as a C1 alcohol such as methanol, a C2 alcohol such as ethanol, a C3 alcohol such as propanol, or a C4 alcohol such as butanol; a C3-C6 ketone such as a C3 ketone such as acetone, a C4 ketone, a C5 ketone, or a C6 ketone, a C2-C6 ether such as C2 ether, a C3 ether, a C4 ether, a C5 ether, or C6 ether; a C3-C5 ester such as a C3 ester, a C4 ester, or a C5 ester such as ethylacetate, a combination of two or more thereof and a combination of one or more thereof with water.
41*. The solid pharmaceutical composition of embodiment 50*, wherein the rapid draying is spray drying.
42*. The solid pharmaceutical composition of any one of embodiments 1* to 41* or 71* to 77*, for use in a method for treating hepatitis C in a human.
43*. The solid pharmaceutical composition of any one of embodiments 1* to 42* or 71* to 77*, for treating hepatitis C in a human.
44*. A process for preparing a solid pharmaceutical composition according to any one of embodiments 1* to 43* or 71* to 77*, comprising
   (i) providing amorphous sofosbuvir.
   (ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
   (iii) processing the mixture obtained from (ii) to the solid pharmaceutical composition.
45*. A process for preparing a solid pharmaceutical composition according to any one of embodiments 1* to 43*, comprising
   (i) providing sofosbuvir by rapid drying, preferably by spray drying a solution comprising sofosbuvir and one or more solvents,
   (ii) mixing the amorphous sofosbuvir of (i) and one or more pharmaceutically acceptable excipient(s), wherein at least 99 weight-% of the mixture consists of the sofosbuvir, and the one or more pharmaceutically acceptable excipient(s);
   (iii) processing the mixture obtained from (ii) to the solid pharmaceutical composition, wherein preferably the solid pharmaceutical composition is a tablet.
46*. The process of embodiment 44* or 45*, wherein at least 99 weight-%, preferably at least 99.5 weight-%, more preferably at least 99.9 weight-% of the sofosbuvir comprised in the solid pharmaceutical composition are present in amorphous form.
47*. The process of any of embodiments 44* to 46*, wherein (iii) preferably consists of (iii)) directly processing the mixture obtained from (ii) to the pharmaceutical composition.
48*. The process of any of embodiments 44* to 47*, wherein after (ii) and before (iii), the mixture obtained from (ii) is not subjected to any modification.
49*. The process of any one of embodiments 44* to 48*, wherein the mixture obtained from (ii) is processed to the pharmaceutical composition according to (iii) at most 168 h, preferably at most 72 h, more preferably at most 24 h after having been obtained from (ii), wherein during this period of time, the mixture is preferably not subjected to stress conditions of 30° C. and a relative humidity of 75%, more preferably stored under ambient conditions.
50*. The process of any one of embodiments 44* to 49*, wherein (iii) comprise one or more of wet granulation, dry granulation, compression, melting extrusion of the mixture of (ii).
51*. The process of embodiment 50*, wherein (iii) comprises direct compression or dry granulation.
52*. The process of any one of embodiments 44* to 51*, wherein (iii) further comprises coating the solid pharmaceutical composition.
53*. The process of any one of embodiments 45* to 52*wherein (i) comprises
   (i′) preparing a solution of sofosbuvir and one or more solvents.
54*. The process of embodiments 45* to 53*, wherein the one or more solvents is selected from the group consisting of an organic solvent, and a combination of two or more thereof.
55*. The process of embodiment 54*, wherein the organic solvent is selected from the group consisting of a C1-C2 halogenated hydrocarbon such as CH₂Cl₂, a C1-C4 alcohol, such as a C1 alcohol such as methanol, a C2 alcohol such as ethanol, a C3 alcohol such as propanol, or a C4 alcohol such as butanol; a C3-C6 ketone such as a C3 ketone such as acetone, a C4 ketone, a C5 ketone, or a C6 ketone; a C2-C6 ether such as C2 ether, a C3 ether, a C4 ether, a C5 ether, or C6 ether; a C3-C5 ester such as a C3 ester, a C4 ester, or a C5 ester such as ethylacetate, a combination of two or more thereof and a combination of one or more thereof with water.
56*. The process of any of embodiments 45* to 54*, wherein the rapid draying is spry drying.
57*. The process of any of embodiments 44* to 55*, wherein the solid pharmaceutical composition comprises the in an amount in the range of from 15 to 95 weight-%, preferably from 20 to 70 weight-%, preferably from 20 to 45 weight-%, more preferably from 20 to 40 weight-%, more preferably from 30 to 40 weight-%, more preferably 30 weight-%, based on the total weight of the solid pharmaceutical composition.
58*. The process of any of embodiments 44* to 57*, wherein from 20 to 45 weight-%, more preferably from 20 to 40 weight-%, more preferably from 30 to 40 weight-%, more preferably 30 weight-%, based on the total weight of the solid pharmaceutical composition.
59*. The process of any of embodiments 44* to 58*, wherein 30 or 35 or 40 or 45 weight-% of the solid pharmaceutical composition consists of sofosbuvir based on the total weight of the solid pharmaceutical composition.
60*. The process of any one of embodiments 44* to 59*, wherein at least 99.5 weight-%, preferably at least 99.9 weight-% of the solid pharmaceutical composition consists of the sofosbuvir and one or more pharmaceutically acceptable excipient(s).
61*. The process of any one of embodiments 44* to 60*, wherein the solid pharmaceutical composition is an oral dosage form selected from the group consisting of a granule, a capsule such as a capsule filled with granules, a sachet, a pellet, a dragee, a lozenge, a troche, a pastille, or a tablet, such as an uncoated tablet, a coated tablet, an effervescent tablet, a soluble tablet, a dispersible tablet, an orodispersible tablet, a tablet for use in the mouth, a chewable tablet and an extrudate, preferably the solid pharmaceutical composition is a tablet or a coated tablet.
62*. The process of any one of embodiments 44* to 61*, wherein the one or more pharmaceutically acceptable excipient(s) of (ii) comprise one or more of at least one of a diluent, of at least one disintegrant, of at least one glidant, of at least one lubricant and combinations of two or more thereof.
63*. The process of embodiment 62*, wherein the at least one diluent includes one or more of calcium carbonate, dicalcium phosphate, dry starch, calcium sulfate, cellulose, compressible sugars, confectioner's sugar, dextrates, dextrin, dextrose, dibasic calcium phosphate dihydrate, glyceryl palmitostearate, hydrogenated vegetable oil, inositol, kaolin, lactose, magnesium carbonate, magnesium oxide, maltodextrin, mannitol, microcrystalline cellulose, polymethacrylates, potassium chloride, powdered cellulose, powdered sugar, pregelatinized starch, sodium chloride, sorbitol, starch, sucrose, sugar spheres, talc, tribasic calcium phosphate, wherein the at least one disintegrant includes one or more of agar, alginic acid, bentonite, carboxymethylcellulose calcium, carboxymethylcellulose sodium, carboxymethylcellulose, cellulose, a cation exchange resin, cellulose, gums, citrus pulp, colloidal silicon dioxide, corn starch, croscarmellose sodium, crospovidone, guar gum, hydrous aluminum silicate, an ion exchange resin such as polyacrin potassium, magnesium aluminum silicate, methyl cellulose, microcrystalline cellulose, modified cellulose gum, modified corn starch, montmorillonite clay, natural sponge, polyacrilin potassium, potato starch, powdered cellulose, povidone, pregelatinized starch, sodium alginate, sodium bicarbonate optionally in admixture with one or more acidulants, sodium starch glycolate, starch, silicates, wherein the at least one glidant includes one or more of colloidal silicon dioxide, talc, starch, starch derivatives, wherein the at least one lubricant includes one or more of calcium stearate, glyceryl monostearate, glyceryl palmitostearate, hydrogenated castor oil, hydrogenated vegetable oil, light mineral oil, magnesium stearate, mineral oil, polyethylene glycol, sodium benzoate, sodium lauryl sulfate, sodium stearyl fumarate, stearic acid, talc, zinc stearate,
64*. The process of any one of embodiments 44* to 63*, wherein the one or more pharmaceutically acceptable excipient(s) of (ii) comprise, preferably are, a combination of mannitol, microcrystalline cellulose, croscarmello se sodium, colloidal silica and magnesium stearate.
65*. The process of any one of embodiments 44* to 64* for preparing the solid pharmaceutical formulation of any one of embodiments 1* to 43* or 71* to 77* or 85*.
66*. A solid pharmaceutical composition obtained or obtainable by a process according to any one of embodiments 44* to 65* or 78* to 84*.
67*. A tablet obtained or obtainable by a process according to any one of embodiments 44* to 66*.
68*. Use of a solid pharmaceutical composition according to any one of embodiments 1* to 43* or 71* to 77* or 85*, for the preparation of a medicament for treating hepatitis C in a human.
69*. A method for treating hepatitis C comprising administering a solid pharmaceutical composition according to any one of embodiments 1* to 43* or 66* or 71* to 77* or 85* to a human in need thereof.
70*. A process for preparing amorphous sofosbuvir comprising spray drying sofosbuvir.
71*. The solid pharmaceutical composition of any of embodiments 1* to 43*, 66*, wherein at least 1.5 weight-% preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
72*. The solid pharmaceutical composition of any one of embodiments 1* to 43*, 66*, 71* wherein at least 15 weight-%, preferably from 20 to 30 weight-%, more preferably from 22 to 26 weight-% of the solid composition consists of the diluent, based on the total weight of the solid pharmaceutical composition.
73*. The solid pharmaceutical composition of any one of embodiments 1* to 43*, 66*, 71*, 72*, wherein at least 16.5 weight-%, preferably from 22 to 36 weight-%, more preferably from 27.5 to 32.5 weight-% of the composition consists of a disintegrant, based on the total weight of the solid pharmaceutical composition.
74*. The solid pharmaceutical composition of any one of embodiments 1* to 43*, 66*, 71* to 73*, wherein at least 15 weight-%, preferably from 20 to 30 weight-%, more preferably from 24 to 28 weight-% of the composition consists of the first disintegrant, based on the total weight of the solid pharmaceutical composition.
75*. The solid pharmaceutical composition of any one of embodiments 1* to 43*, 66*, 71* to 74*, wherein at least 1.5 weight-%, preferably from 2 to 6 weight-%, more preferably from 2.5 to 5.5 weight-% of the composition consists of the second disintegrant, based on the total weight of the solid pharmaceutical composition.
76*. The solid pharmaceutical composition of any one of embodiments 1* to 43*, 66*, 71* to 75*, wherein at least 0.2 weight-%, preferably from 0.5 to 7 weight-%, more preferably from 0.8 to 5 weight-% of the composition consists of the glidant, based on the total weight of the solid pharmaceutical composition.
77*. The solid pharmaceutical composition of any one of embodiments 1* to 43*, 66*, 71* to 76*, wherein at least 0.5 weight-%, preferably from 1 to 6 weight-%, more preferably from 1.4 to 2 weight-% of the composition consists of the lubricant, based on the total weight of the solid pharmaceutical composition.
78*. The process of any of embodiments 44* to 65*, wherein at least 1.5 weight-% more preferably from 10 to 85 weight-%, more preferably from to 30 to 80 weight-%, more preferably from 40 to 70 weight-%, more preferably from 50 to 60 weight-% of the solid pharmaceutical composition consists of the one or more pharmaceutically acceptable excipients based on the total weight of the solid pharmaceutical composition.
79*. The process of any one of embodiments 44* to 65*, 78*, wherein at least 15 weight-%, preferably from 20 to 30 weight-%, more preferably from 22 to 26 weight-% of the solid composition consists of the diluent, based on the total weight of the solid pharmaceutical composition.
80*. The process of any one of embodiments 44* to 65*, 78*, 79*, wherein at least 16.5 weight-%, preferably from 22 to 36 weight-%, more preferably from 27.5 to 32.5 weight-% of the composition consists of a disintegrant, based on the total weight of the solid pharmaceutical composition.
81*. The process of any one of embodiments 44* to 65*, 78* to 80*, wherein at least 15 weight-%, preferably from 20 to 30 weight-%, more preferably from 24 to 28 weight-% of the composition consists of the first disintegrant, based on the total weight of the solid pharmaceutical composition.
82*. The process of any one of embodiments 44* to 65*, 78* to 81*, wherein at least 1.5 weight-%, preferably from 2 to 6 weight-%, more preferably from 2.5 to 5.5 weight-% of the composition consists of the second disintegrant, based on the total weight of the solid pharmaceutical composition.
83*′. The process of any one of embodiments 44* to 65*, 78* to 82*, wherein at least 0.2 weight-%, from 0.5 to 7 weight-%, more preferably from 0.8 to 5 weight-% of the composition consists of the glidant, based on the total weight of the solid pharmaceutical composition.
84*. The process of any one of embodiments 44* to 65*, 78* to 83*, wherein at least 0.5 weight-%, preferably from 1 to 6 weight-%, more preferably from 1.4 to 2 weight-% of the composition consists of the lubricant, based on the total weight of the solid pharmaceutical composition.
85* The solid pharmaceutical composition of any one of embodiments 1* to 43*, 66*, 71* to 77*, wherein the solid pharmaceutical composition is a solid dispersion.
86*. The process of any one of embodiments 44* to 65*, 78* to 83* or 85*, wherein the solid pharmaceutical composition is a solid dispersion.

The present invention is further illustrated by the following reference examples and examples.

### EXAMPLES

### Example 1: Preparation of a Solid Pharmaceutical Composition Comprising Amorphous Sofosbuvir: Hot Melt Extrusion

400 mg sofosbuvir crystalline form 1 prepared according to WO 2011/123645 A, Example 10.90 mg Kollidon® (a vinyl pyrrolidone vinyl acetate copolymer; from BASF SE), 270 mg of mannitol powder, 300 mg of cellulose microcrystalline 102, 30 mg of croscarmellose sodium and 54 mg of colloidal silica dioxide were dry-mixed.

The obtained dry mixture was subjected to hot-melt extrusion at (120±30) ° C. using a DSM-Explore V micro compounder. The obtained extrudate was cooled to ambient temperature (25° C.). The cooled extrudate was then crushed using a sieve having a mesh size of 1 mm.

The extrudate was farther mixed with 60 mg of Cellulose Microcrystalline, 30 mg Croscarmellose sodium 6 mg colloidal silicon dioxide and 9 mg of Magnesium stearate. The mixture was further tableted by direct compression.

The tablet was coated with Opadry II

TABLE 1
Mixture according to Example 1

| | Amount |
|---|---|
| Component | in mg |
| Sofosbuvir | 400 |
| Kollidon ® | 90 |
| Mannitol | 270 |
| Microcrystalline Cellulose (Avicel ® 102) | 300 |
| Crosscarmellose Sodium (Ac-Di-Sol ® Typ A) | 30 |
| Silica dioxide | 54 |
| Extrudate | 1095 |
| Microcrystalline Cellulose | 60 |
| Crosscarmellose Sodium | 30 |
| Colloidal silicon dioxide | 6 |
| Magnesium stearate | 9 |
| Opadry II | 35 |
| E-Water | 140 |

The tablets were tested in dogs. The tablets show excellent pharmacokinetic properties in terms of Cₘₐₓ and t_{1/2}.

### Example 2: Preparation of a Solid Dispersion Comprising Amorphous Sofosbuvir and a Silicon-Based Inorganic Adsorbent as the Matrix Compound: Spray Dry

400 mg sofosbuvir crystalline Form 1 prepared according to WO 2011/123645 A, Example 10, were dissolved in acetone (65% W/W). Syloid® 72FP silica (a synthetic amorphous silica; from Grace) was added (25% on API W/W). The suspension was spray dried at 65° C. under 40 mm Nitrogen flow, at a spray rate of 13/ml/min. After spray drying mannitol (300 mg), microcrystalline cellulose 102, croscarmellose sodium, colloidal silicon dioxide and magnesium stearate were added. The blend was dried and compacted. Microcrystalline cellulose 102, croscarmellose sodium, colloidal silicon dioxide and magnesium stearate was added and blended. The blend was tableted and the cores were coated using Opadry II solution (35 mg)

The amounts used are reported in the table below:

| Component | Amount/mg |
|---|---|
| Intragranular | |
| Sofosbuvir spray dried with | 533.33 ¹⁾²⁾³⁾ |
| Syloid (solid dispersion) corresponding | |
| to 400 mg sofosbuvir) | |
| Microcrystalline Cellulose | 256.1 |
| (Avicel ® 102) | |
| Pearlitol 100 SD (mannitol) | 300.00²⁾³⁾ |
| Ac-Di_Sol (Croscarmellose Sodium) | 30.00 |
| Aerosil (colloidal silicon dioxide) | 54.00 |
| Magnesium Stearate | 90 |
| Extargranular | |
| Cellulose MicroKrist. | 60 |
| Intragranular | |
| Ac-Di_Sol (Croscarmellose Sodium) | 30 |
| Aerosil (colloidal silicon dioxide) | 6 |
| Magnesium Stearate | 9 |
| Coating | |
| Opadry II | 35 |
| E-water | 140 |

¹⁾ Drug is used in the form of spray dired intermeditae
²⁾Varies with the intial mass of the drug substance
³⁾based on the content of 75% in the spraydried intemediate the amount of Mannitol 100SD (Pearlitol 100SD) is 249.67 mg.

The tablets were tested in dogs. The tablets show excellent pharmacokinetic properties in terms of Cₘₐₓ and t_{1/2}.

### Example 3: Preparation of a Solid Dispersion Comprising Amorphous Sofosbuvir: Spray Dry

400 mg sofosbuvir crystalline Form 1 prepared according to WO 2011/123645 A, Example 10 was spray dried at 65° C. under 40 mm Nitrogen flow, at a spray rate of 13/ml/min. After spray drying mannitol, microcrystalline cellulose 102, croscarmellose sodium, colloidal silicon dioxide and magnesium stearate are added. The blend was dried and dry granulated. Microcrystalline cellulose 102 croscarmellose sodium colloidal silicon dioxide and magnesium stearate was added and blended. The blend was tableted and the cores were coated using Opadry II solution.

The amounts used are reported in the table below:

| Components | Amount/mg |
|---|---|
| Sofosbuvir pure | 400.00 ¹⁾²⁾³⁾ |
| Cellulose MicroKrist. | 296.1 |
| Pearlitol 100 SD (mannitol) | 360.00²⁾³⁾ |
| Ac-Di_Sol (Croscarmellose Sodium) | 30.00 |
| Aerosil (colloidal silicon dioxide) | 54.00 |
| Magnesium Stearate | 90 |
| Cellulose MicroKrist. | 60 |
| Ac-Di_Sol (Croscarmellose Sodium) | 30 |
| Aerosil (colloidal silicon dioxide) | 6 |
| Magnesium Stearate | 9 |
| Coating | |
| Opadry II | 35 |
| E-water | 140 |

¹⁾ Drug is used in the form of spray dired intermeditae
²⁾Varies with the intial mass of the drug substance
³⁾based on the content of 100% in the spraydried intemediate the amount of Mannitol 100SD (Pearlitol 100 SD) is 330.00 mg.

The tablets were tested in dogs. The tablets show excellent pharmacokinetic properties in terms of Cₘₐₓ and t_{1/2}.

### Example 4: Preparation of a Solid Dispersion Comprising Amorphous Sofosbuvir: Spray Dry

400 mg sofosbuvir amorphous form was added to a first portion of mannitol, microcrystalline cellulose 102, croscarmellose sodium, colloidal silicon dioxide and magnesium stearate. The blend was sieved on a 0.5 mm sieve and mixed in a fall blender. The material was compacted using a roller compactor (Main Pressure 100 bar, gap 2-3 mm, Speed 8 rpm, first sieve 2.0 mm, second sieve 1.0 mm).

A second portion of microcrystalline cellulose 102, croscarmellose sodium, colloidal silicon dioxide was sieved on a 0.5 mm sieve and mixed in a fall blender together with the roller compacted mixture. Magnesium stearate was added 3 minutes before the end. The blend was tabled and coated by spray dry using Opadry II solution (20% Opadry and 80% water).

The amounts used are reported in the table below:

| Components | Amount/mg |
|---|---|
| Amorphous Sofosbuvir pure | 400 |
| Cellulose MicroKrist. | 280 |
| Pearlitol 100 SD (mannitol) | 330 |
| Ac-Di_Sol (Croscarmellose Sodium) | 30 |
| Aerosil (colloidal silicon dioxide) | 6 |
| Magnesium Stearate | 9 |
| Cellulose MicroKrist. | 65 |
| Ac-Di_Sol (Croscarmellose Sodium) | 30 |
| Aerosil (colloidal silicon dioxide) | 6 |
| Magnesium Stearate | 9 |
| Coating | |
| Opadry II | 35 |

### CITED PRIOR ART

WO 2013/101550 A
WO 2011/123645 A

1. A solid pharmaceutical composition wherein the solid pharmaceutical composition comprises sofosbuvir according to formula (I) 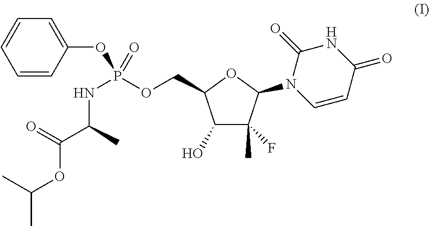 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir and the one or more pharmaceutically acceptable excipient(s).

2. A solid pharmaceutical composition wherein the solid pharmaceutical composition comprises sofosbuvir according to formula (I) 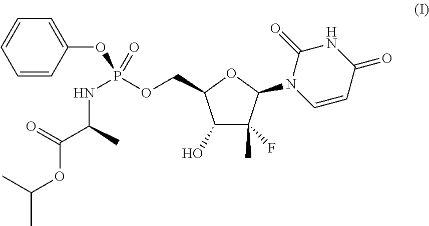 [SMILES: CC(C)OC(=O)[C@H](C)N[P@](=O)(OC[C@H]1O[C@@H](n2ccc(=O)[nH]c2=O)[C@](C)(F)[C@@H]1O)Oc1ccccc1]
at least one pharmaceutically acceptable matrix compound and one or more pharmaceutically acceptable excipient(s), wherein at least 99 weight-% of the sofosbuvir comprised in the solid pharmaceutical composition are present in amorphous form, and at least 99 weight-% of the solid pharmaceutical composition consists of the sofosbuvir, the at least one matrix compound and the one or more pharmaceutically acceptable excipient(s).

3. The solid pharmaceutical composition claim 1, wherein the solid pharmaceutical composition is a solid dispersion.

4. The solid pharmaceutical composition of claim 1, wherein the solid pharmaceutical composition comprises the sofosbuvir in an amount in the range of from 15 to 95 weight-%.

5. The solid pharmaceutical composition of claim 1, wherein the solid pharmaceutical composition comprises the sofosbuvir in an amount in the range from 20 to 40 weight-%, based on the total weight of the solid pharmaceutical composition.

6. The solid pharmaceutical composition of claim 2, wherein the at least one matrix compound is selected from the group consisting of hydrophilic polymers, silicon-based inorganic adsorbents and a combination of two or more thereof.

7. The solid pharmaceutical composition of claim 6, wherein the silicon-based inorganic adsorbents include one or more of silica and silicates.

8. The solid pharmaceutical composition of claim 7, wherein the hydrophilic polymers include one or more of polysaccharides, polyvinylpyrrolidones, polyethylene glycols, polyethylene glycol based copolymers, polyacrylic acids, salts of polyacrylic acids, polyvinyl alcohols, polyacrylamide copolymers, methacrylic acid copolymers, methacrylate copolymers, pectines, chitin derivatives, chitosan derivatives, polyphosphates, polyoxazolines.

9. The solid pharmaceutical composition of claim 8, wherein the hydrophilic polymers include copovidone.

10. The solid pharmaceutical composition of claim 1, wherein at least 1.5 weight-% of the solid pharmaceutical composition consists of the one or more pharmaceutically acceptable excipients based on the total weight of the solid pharmaceutical composition.

11. The solid pharmaceutical composition of claim 1, obtainable or obtained by a process comprising
(i) preparing amorphous sofosbuvir;
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

12. The solid pharmaceutical composition of claim 10, wherein the preparing of (i) is carried out by rapid drying a solution comprising sofosbuvir and one or more solvents.

13. The solid pharmaceutical composition of claim 2, obtainable or obtained by a process comprising
(i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
(ii) processing the mixture obtained from (i) to the solid pharmaceutical composition.

14. The solid pharmaceutical composition of claim 13, wherein (ii) comprise one or more of wet granulation, dry granulation, compression, melting extrusion of the mixture of (i).

15. The solid pharmaceutical composition of claim 2, obtainable or obtained by a process comprising
(i′) preparing a mixture of sofosbuvir and the at least one pharmaceutically acceptable matrix compound and at least one solvent;
(ii′) subjecting the mixture of (i′) to drying, obtaining a dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition.

16. The solid pharmaceutical composition of claim 1, wherein the solid pharmaceutical composition is a tablet.

17. A process for preparing a solid pharmaceutical composition according to claim 1, the process comprising:
(i) preparing amorphous sofosbuvir,
(ii) mixing the amorphous sofosbuvir of (i) and one or more pharmaceutically acceptable excipient(s), wherein at least 99 weight-% of the mixture consists of the sofosbuvir and the one or more pharmaceutically acceptable excipient(s);
(iii) processing the mixture obtained from (ii) to the solid pharmaceutical composition.

18. The process of claim 17 wherein the preparing of (i) is carried out by rapid drying a solution comprising sofosbuvir and one or more solvents.

19. A process for preparing a solid pharmaceutical composition, the process comprising:
(i) preparing a mixture comprising sofosbuvir, at least one pharmaceutically acceptable matrix compound and one or more pharmaceutically acceptable excipient(s), wherein at least 99 weight-% of the mixture consists of the sofosbuvir, the at least one matrix compound and the one or more pharmaceutically acceptable excipient(s);
(ii) processing the mixture obtained from (i) to the solid pharmaceutical composition.

20. The process of claim 19, wherein (ii) comprise one or more of wet granulation, dry granulation, compression, melting extrusion of the mixture of (i).

21. A process for preparing a solid pharmaceutical composition, the process comprising:
(i′) preparing a mixture of sofosbuvir and the at least one pharmaceutically acceptable matrix compound and at least one solvent;
(ii′) subjecting the mixture of (i′) to drying the mixture of (i′), obtaining a dried mixture-1;
(iii′) mixing the mixture-1 of (ii′) with the one or more pharmaceutically acceptable excipient(s), obtaining a mixture-2;
(iv′) processing the mixture-2 of (iii′) to the solid pharmaceutical composition.
